(19) United States
(12) Patent Application Publication
(10) Pub. No.: US 2023/0420080 A1
(43) Pub. Date: Dec. 28, 2023

(54) SPLIT-READ ALIGNMENT BY INTELLIGENTLY IDENTIFYING AND SCORING CANDIDATE SPLIT GROUPS

(71) Applicant: Illumina Software, Inc., San Diego, CA (US)

(72) Inventor: Michael Ruehle, Fort Worth, TX (US)

(21) Appl. No.: 18/340,795

(22) Filed: Jun. 23, 2023

Related U.S. Application Data
(60) Provisional application No. 63/367,002, filed on Jun. 24, 2022.

(51) Int. Cl. G16B 40/00; G16B 30/10

(52) U.S. Cl. CPC G16B 40/00; G16B 30/10

(57) ABSTRACT

The present disclosure relates to systems, non-transitory computer-readable media, and methods for efficiently identifying and selecting split groups corresponding to one or more nucleotide reads. Generally, split groups comprise chains of fragments forming split-alignments of one read. The disclosed system utilizes dynamic programming to generate and evaluate candidate split groups. The disclosed system can generate split group scores for each of the candidate split groups. To generate the split group scores, the disclosed system considers fragment alignment scores and geometries of fragment alignments within the candidate split groups. The disclosed systems select a predicted split group from the candidate split groups based on the split group scores.

### CROSS-REFERENCE TO RELATED APPLICATIONS

The present application claims the benefit of, and priority to, U.S. Provisional Application No. 63/367,002, entitled “IMPROVING SPLIT-READ ALIGNMENT BY INTELLIGENTLY IDENTIFYING AND SCORING CANDIDATE SPLIT GROUPS,” filed on Jun. 24, 2022. The aforementioned application is hereby incorporated by reference in its entirety.

### BACKGROUND

In recent years, biotechnology firms and research institutions have improved hardware and software for sequencing nucleotides and determining nucleobase calls for genomic samples. For instance, some existing sequencing machines and sequencing-data-analysis software (together “existing sequencing systems”) predict individual nucleobases within sequences by using conventional Sanger sequencing or sequencing-by-synthesis (SBS) methods. When using SBS, existing sequencing systems can monitor many thousands of oligonucleotides being synthesized in parallel from templates to predict nucleobase calls for growing nucleotide reads. A camera in many existing sequencing systems captures images of irradiated fluorescent tags incorporated into oligonucleotides. After capturing such images, some existing sequencing systems determine nucleobase calls for nucleotide reads corresponding to the oligonucleotides and send base-call data to a computing device with sequencing-data-analysis software, which aligns nucleotide reads with a reference genome. Based on differences between the aligned nucleotide reads and the reference genome, existing systems (e.g., a variant caller) determines nucleobase calls for genomic regions and identify variants of a genomic sample.

Despite these recent advances, existing sequencing systems often inaccurately identify and align split reads with a reference genome and, consequently, fail to determine variant or other nucleobase calls or determine inaccurate nucleobase calls. Generally, a split read represents a nucleotide read that has one read fragment that maps to (or aligns with) one region of a reference genome and one or more other read fragments that map to (or aligns with) different regions of the reference genome. For example, a nucleotide read that covers a structural variant, different sides of a deletion, different sides of a gene fusion, or simply random mapping of read fragments can result in a split read. Indeed, in a split read, one read fragment from a nucleotide read may align best to a genomic region on one chromosome and another read fragment from the same nucleotide read may align best with a genomic region on another chromosome. Because such a split-read alignment on two different chromosomes (or different genomic regions on a same chromosome) may either accurately reflect a variant of a genomic sample or erroneously suggest a split read that should align to a single genomic region, existing sequencing systems have developed computational models to recognize and distinguish between correct and incorrect split-read alignments.

While existing computational models can accurately recognize some split-read alignments, such computational models include design flaws that routinely lead to misidentifying split-read alignments. For example, some existing sequencing systems determine a primary alignment for a split read based on a highest scoring alignment of a single read fragment from candidate alignments of candidate read fragments. But such existing sequencing systems fail to consider split-alignment possibilities and account for how alignments of multiple fragments together score relative to other candidate alignments. To further illustrate, many existing sequencing systems determine a primary alignment that clips read fragments (or the different ends of a read) and thereby leave a gap between fragment alignments. To fill such a gap, some existing sequencing systems iteratively select additional fragment alignments that overlap with the gap. By merely plugging gaps without considering fragment alignments together, such existing systems fail to consider the relative fragment positions or orientations or other split-alignment geometries of a nucleotide read relative to a reference genome.

Due in part to inaccuracies of aligning read fragments, existing sequencing systems often determine inaccurate variant calls or other base calls based on inaccurate split-read alignments. For example, by prioritizing a primary alignment without considering fragment alignments from a nucleotide read as a whole, some existing sequencing systems may incorrectly disregard a fragment alignment that correctly reflects a structural variant and fills in gaps indicative of a deletion together with other fragment alignments. Conversely, a primary alignment of a read fragment may by itself map best to an incorrect genomic region of a reference genome. By prioritizing the primary alignment, some existing sequencing systems disregard a correct genomic region better reflected by alignments of multiple fragments from a nucleotide read, thereby resulting in a false-negative variant call or an otherwise incorrect variant call. Thus, existing sequencing systems frequently misalign, incorrectly match, or miss call variants for a large number of samples as well as increase the chances of mismatched alignments with reads from a genomic sample.

To compensate for the failure of some existing sequencing systems to correctly detect split-read alignments indicating structural variants, some existing systems perform both whole genome sequencing (WGS) using SBS (or other techniques) and microarrays with genotyping probes that target specific structural variants. Indeed, microarrays have been specifically designed to target hard-to-detect structural variants using existing sequencing devices. By running both WGS and multiple microarrays—and sometimes using different specialized sequencing devices and microarray devices—existing sequencing systems multiply the computer processing and time to determine accurate variant calls for both single nucleotide polymorphisms (SNPs) and smaller insertions and deletions (indels), on the one hand, and structural variants, on the other hand.

### BRIEF SUMMARY

This disclosure describes implementations of methods, non-transitory computer-readable media, and systems that can solve one or more of the foregoing (or other problems) in the art. For example, the disclosed systems can determine scores for alignments of one or more fragments from a nucleotide read in candidate split groups and select a predicted split group from among the candidates based on such scores to use for base calling. In particular, the disclosed systems can identify fragment alignments comprising candidate local alignments of fragments of a read from a genomic sample with a reference genome. The disclosed systems then group such fragment alignments into candidate split groups and determine split group scores for each of these candidate split groups. Based on the split group scores, the disclosed systems identify a predicted split group from among the candidate split groups to use for base calling.

Additional features and advantages of one or more implementations of the present disclosure are outlined in the description which follows, and in part will be obvious from the description, or may be learned by the practice of such example implementations.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The detailed description provides one or more implementations with additional specificity and detail through the use of the accompanying drawings, as briefly described below.

FIG. 1 illustrates a diagram of an environment in which a split-read alignment system can operate in accordance with one or more implementations.

FIG. 2 illustrates an overview of the split-read alignment system determining scores for alignments of one or more fragments from a nucleotide read in a candidate split group and selecting a predicted split group from among the candidates based on such scores to use for base calling in accordance with one or more implementations.

FIGS. 3A-3B illustrate an overview of the split-read alignment system generating candidate split groups for single-end and paired-end nucleotide reads in accordance with one or more implementations.

FIG. 4 illustrates the split-read alignment system utilizing dynamic programming to generate and evaluate candidate split groups in accordance with one or more implementations.

FIG. 5 illustrates an overview of the split-read alignment system generating a split group score in accordance with one or more implementations.

FIGS. 6A-6B illustrate the split-read alignment system determining pair scores and selecting a predicted split group based on the pair scores in accordance with one or more implementations.

FIG. 7 illustrates split-read alignment system generating an alt-contig fragment alignment score for an alignment between read fragments and an alternate contiguous sequence and selecting the alt-contig fragment alignment as a replacement split group score in accordance with one or more implementations.

FIG. 8 illustrates the split-read alignment system utilizing a threshold fragment alignment score to remove candidate split groups in accordance with one or more implementations.

FIG. 9 illustrates the split-read alignment system utilizing a minimum alignment score to identify candidate split groups on which to not report alignments in accordance with one or more implementations.

FIG. 10 illustrates the split-read alignment system generating a variant call for a genomic sample based on predicted split groups in accordance with one or more implementations.

FIGS. 11A-11D illustrate read pile-ups within a graphical user interface showing that the split-read alignment system determines true-negative variant calls for candidate gene fusion events in an improvement over an existing sequencing system that incorrectly determines such variant calls as gene fusion events in accordance with one or more implementations.

FIGS. 12A-12D illustrate coverage graphs exhibiting higher coverage of nucleotide reads mapped and aligned to genomic regions of chromosome M using the split-read alignment system relative to such coverage from nucleotide reads mapped and aligned using an existing sequencing system in accordance with one or more implementations.

FIG. 13 illustrates a variant-call table exhibiting better accuracy for SNP calls and indel calls by the split-read alignment system at genomic regions of chromosome M relative to such SNP calls and indel calls by an existing sequencing system in accordance with one or more implementations.

FIGS. 14A-14B illustrate tables exhibiting improved accuracy of structural variant calls by the split-read alignment system relative to an existing sequencing system in accordance with one or more implementations.

FIG. 15 illustrates a flowchart of a series of acts for determining candidate split groups and selecting a predicted split group based on split group scores in accordance with one or more implementations.

FIG. 16 illustrates a block diagram of an example computing device for implementing one or more implementations of the present disclosure.

### DETAILED DESCRIPTION

This disclosure describes one or more implementations of a split-read alignment system that can select a split group from among candidate split groups of read fragment alignments based on generating and scoring such candidate split groups. Generally, the split-read alignment system identifies a single-end read or paired-end reads corresponding to a genomic sample's genomic region and analyzes candidate split groups comprising alignments of one or more read fragments together rather than finding a single fragment in isolation with a highest alignment score. More specifically, the split-read alignment system can identify candidate local alignments of fragments of a read and create chains of fragment alignments into candidate spit groups. The split-read alignment system scores the candidate split groups and selects a predicted split group for base calling based on the candidate split group scores.

As mentioned, the split-read alignment system can determine candidate split groups. Generally, a candidate split group can comprise (i) one or more fragment alignments of a single-end nucleotide read or (ii) one or more fragment alignments from a paired-end nucleotide read from a pair of paired-end nucleotide reads. In some embodiments, the split-read alignment system efficiently determines the candidate split groups by using dynamic programming. Generally, in dynamic programming, instead of considering every possible combination of fragment alignments, the split-read alignment system iterates from outermost fragment alignments to innermost fragment alignments to determine split groups and split group scores. By using dynamic programming, the split-read alignment system effectively considers all possible or likely combinations of fragment alignments from a nucleotide read.

The split-read alignment system can further generate split group scores for fragment alignments of the candidate split groups. Generally, a split group score indicates a likelihood of fragment alignments in a candidate split group representing correct alignments with a reference genome. The split group scores account for the possibility of split-alignments and split-alignment geometry. Thus, by determining split group scores rather than merely alignment scores for isolated fragment alignments, the split-read alignment system improves the likelihood of choosing a correct fragment alignment or combination of fragment alignments to complete a template.

In some implementations, the split-read alignment system generates a split group score for a candidate split group based on one or more of (i) fragment alignment scores, (ii) a break penalty, (iii) an overlap penalty, or other penalties for fragment alignments within the candidate split group. As part of the split group score, for instance, the split-read alignment system determines fragment alignment scores for individual fragments of the candidate split group. As an additional part of the split group score, in some embodiments, the split-read alignment system determines a break penalty for relative geometries of fragment alignments within the candidate split group (e.g., to penalize breaks between fragment alignments). As yet another part of the split group score, in certain implementations, the split-read alignment system determines an overlap penalty for overlap between or among fragment alignments within the candidate split group. As described below, the split-read alignment system can combine (i), (ii), and (iii) to determine a split group score.

For paired-end nucleotide reads, the split-read alignment system may also identify and score candidate pairs of split groups. Generally, in certain implementations, the split-read alignment system further considers and determines pair scores for paired-end mates to identify a likely split group from among candidate split groups of paired-end mates. For instance, the split-read alignment system can sum split group scores for respective candidate pairs of split groups from a paired-end mate and estimate an insert size between innermost fragment alignments of the candidate pairs of split groups. The split-read alignment system can then generate a pair score for a candidate pair of split groups based on the summed split group scores and the estimated insert size. To illustrate, the split-read alignment system can include a pair score penalty for less likely estimated insert sizes.

In addition to scoring and selecting split groups, in some embodiments, the split-read alignment system can further identify fragment alignments that align with alternate contiguous sequences within a reference genome by using split groups to report a corresponding split alignment. When the split-read alignment system determines that a nucleotide read aligns best to an alternate contiguous sequence based on split-group scoring, in some embodiments, the split-read alignment system reports a split alignment in the primary assembly corresponding to the alternate contiguous sequence by a liftover relationship. For instance, in some cases, the split-read alignment system determines an alt-contig fragment alignment score for fragment alignments corresponding to a nucleotide read with an alternate contiguous sequence representing a structural variant. The split-read alignment system can also determine a split group score for a corresponding split alignment of the fragment alignments with the primary assembly of the reference genome. The split-read alignment system can utilize a higher-scoring alt-contig fragment alignment score as a replacement split alignment score to guide selection of the corresponding split group over other candidate split groups. If the alt-contig fragment alignment score exceeds split group scores for other candidate split groups, for example, the split-read alignment system selects and reports the split alignment with the primary assembly corresponding to the alternate contiguous sequence rather than split alignments represented by the other candidate split groups that may have otherwise scored better in the absence of the alt-contig fragment alignment score.

Based on one or both of the split group scores and pair scores, as mentioned, the split-read alignment system selects a predicted split group from the candidate split groups to use for nucleobase calling. For instance, in some embodiments, the split-read alignment system selects a predicted split group with a highest split group score for each mate of a nucleotide-read pair. In another example, the split-read alignment system selects a predicted split group for each mate of a nucleotide-read pair in accordance with the highest pair score among all pair scores generated from pairs of scored split groups. As a result of selecting a predicted split group, the split-read alignment system improves the accuracy of nucleobase calls and predicted variant calls in output files (e.g., variant call files).

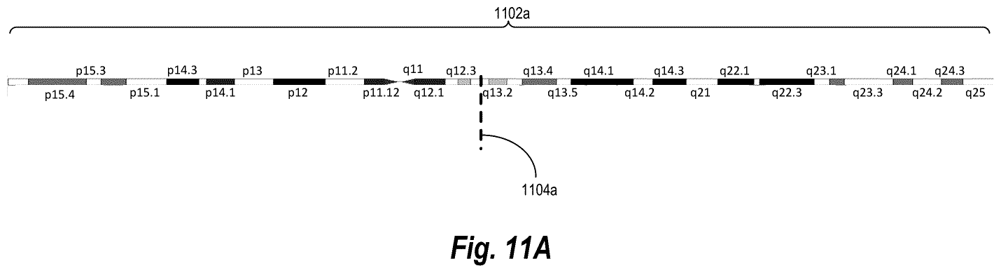

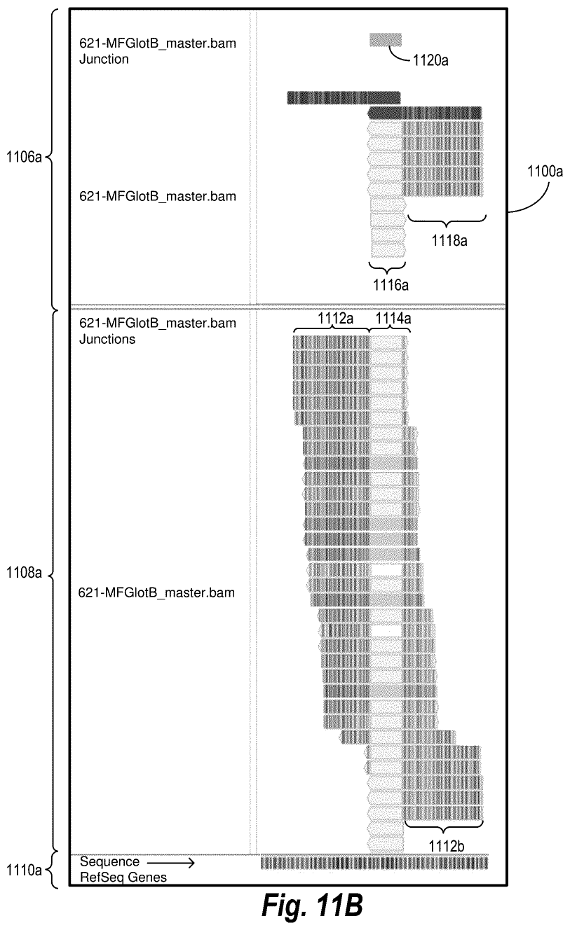

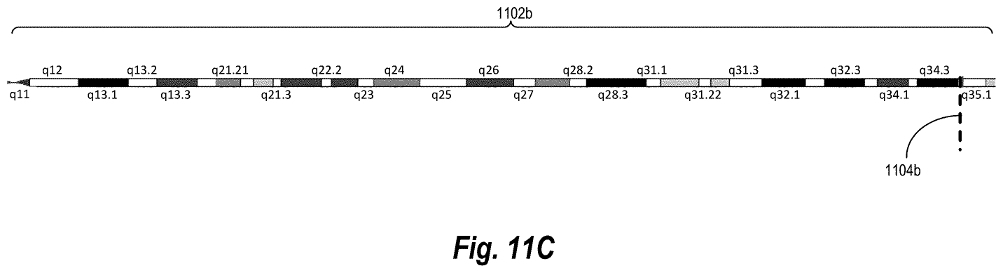

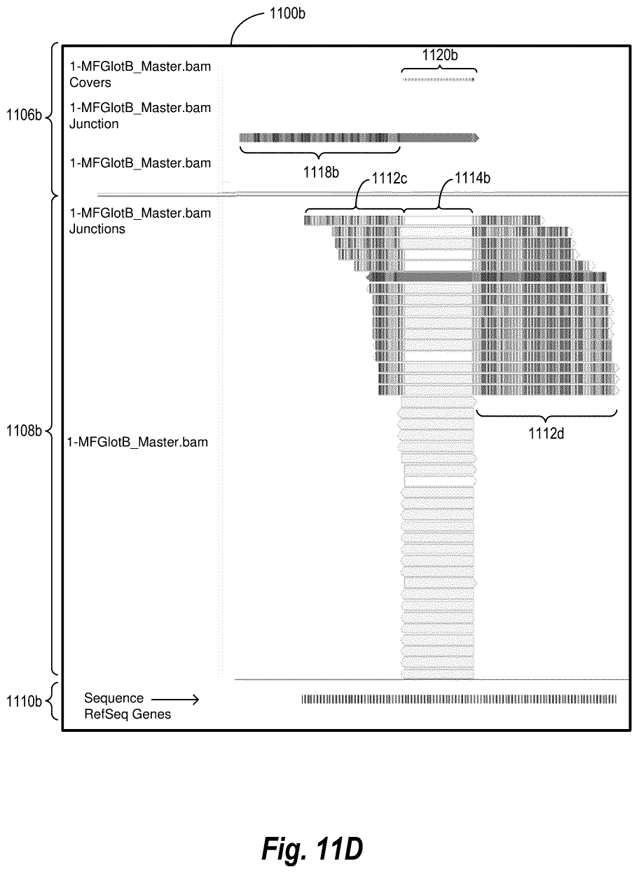

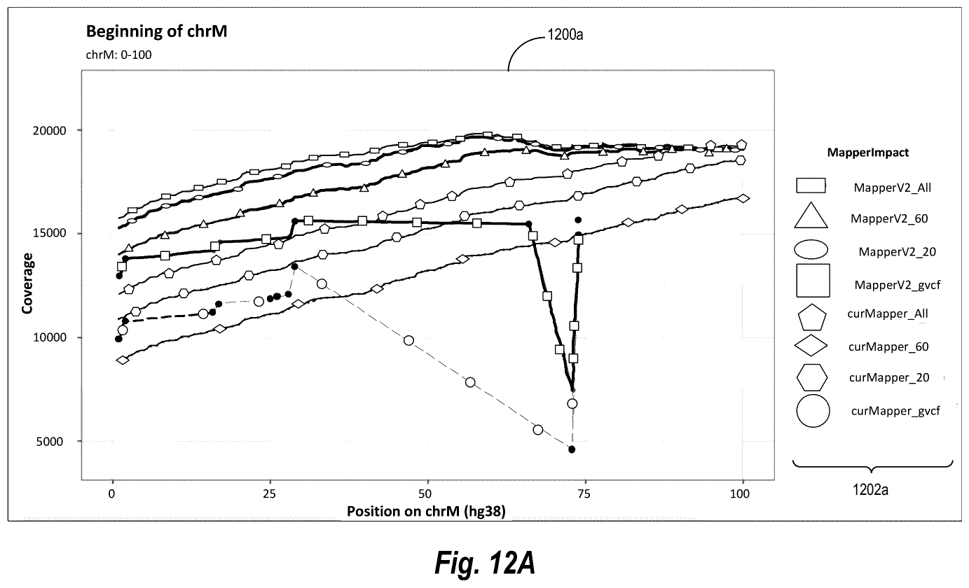

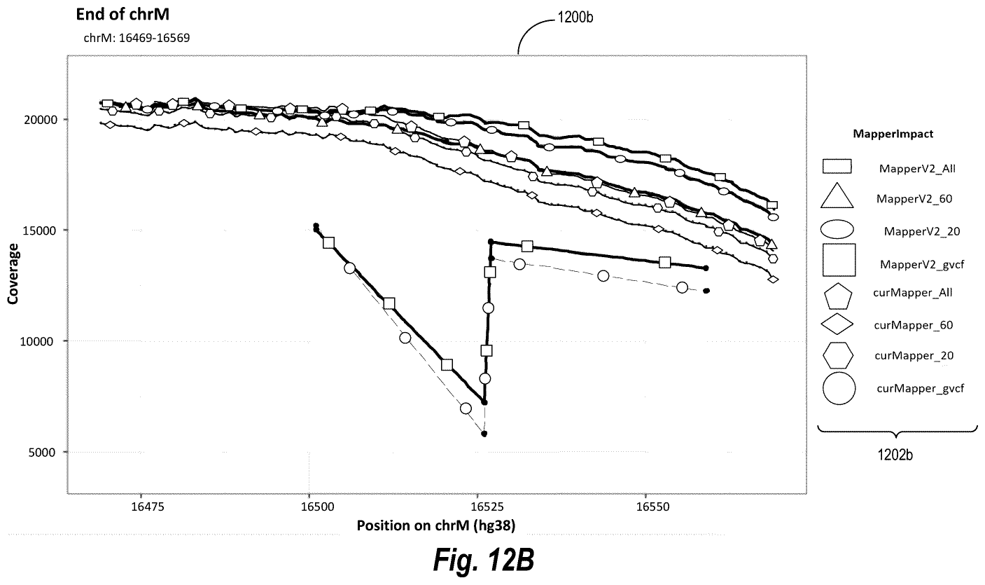

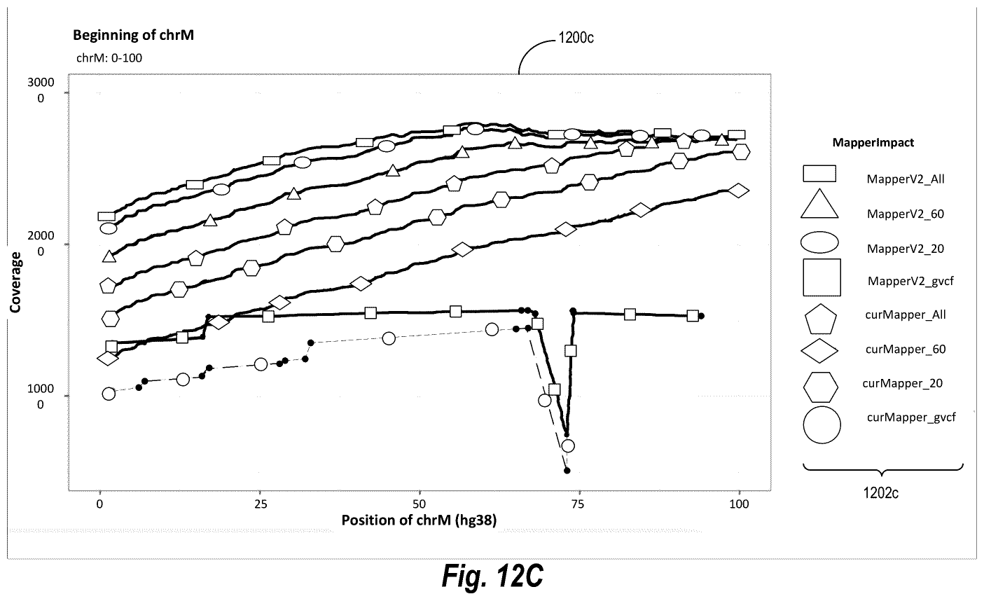

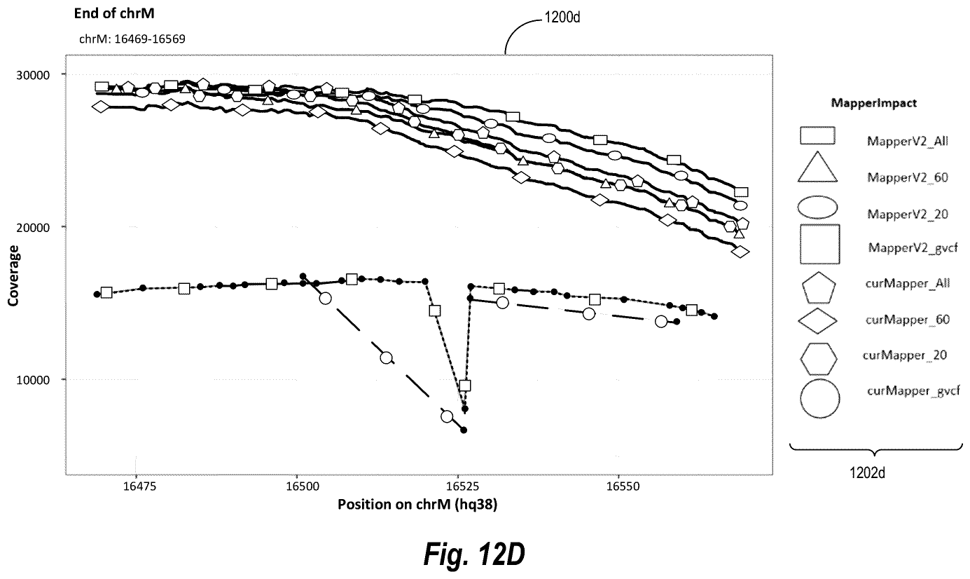

As just suggested above, the split-read alignment system provides several technical advantages and benefits over existing sequencing systems and methods. For example, the split-read alignment system improves the alignment accuracy of split reads over existing sequencing systems by considering split-alignment possibilities within various candidate split groups corresponding to a nucleotide read. By determining split group scores for candidate split groups comprising fragment alignments from fragments of a nucleotide read and selecting a predicted split group from among the candidates based on such split group scores, the split-read alignment system identifies fragment alignments for a split read with better accuracy than existing sequencing systems. As illustrated by FIGS. 11A-11D, for example, the split-read alignment system determines better mappings and alignments for transcriptomic reads and determines more accurate true-negative variant calls for candidate gene fusion events than an existing sequencing system. As shown in FIGS. 12A-12D, the split-read alignment system also determines better mappings and alignments for nucleotide reads on genomic regions of chromosome M for mitochondrial DNA resulting in improved coverage relative to existing sequencing systems. Rather than merely finding a primary alignment for a single fragment with a highest alignment score, the split-read alignment system considers and scores candidate fragment alignments from a nucleotide read together as part of a split group.

In addition to considering fragment alignments together rather than in isolation, in certain implementations, the split-read alignment system also improves the accuracy of split-read alignments with other computational model improvements. For a given split group, for instance, the split-read alignment system determines a break penalty for relative geometries of fragment alignments in a candidate split group. In some cases, the split-read alignment system efficiently identifies and scores such split groups—and quickly identifies a likely split-read alignment—by utilizing dynamic processing to exhaustively consider candidate split groups. For each candidate split group, in some embodiments, the split-read alignment system generates a split group score based on fragment alignment scores, a break penalty, and an overlap penalty, thereby wholistically evaluating the likelihood of a given candidate split group comprising fragment alignments.

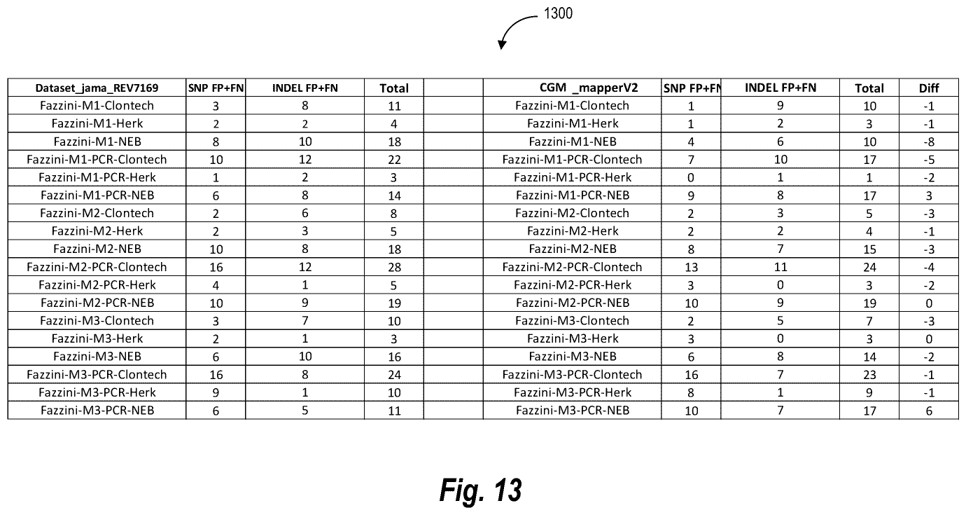

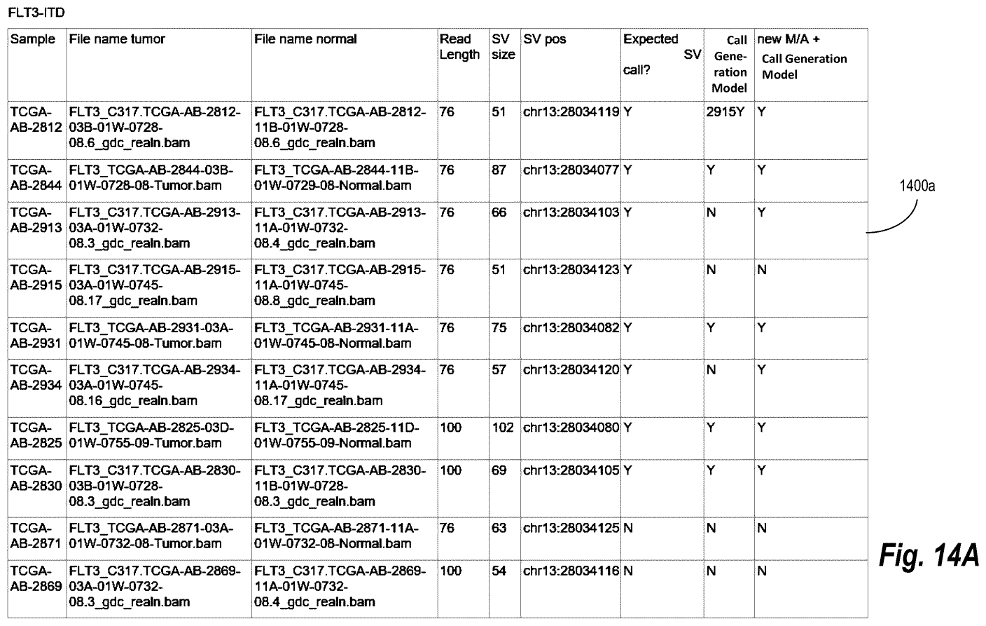

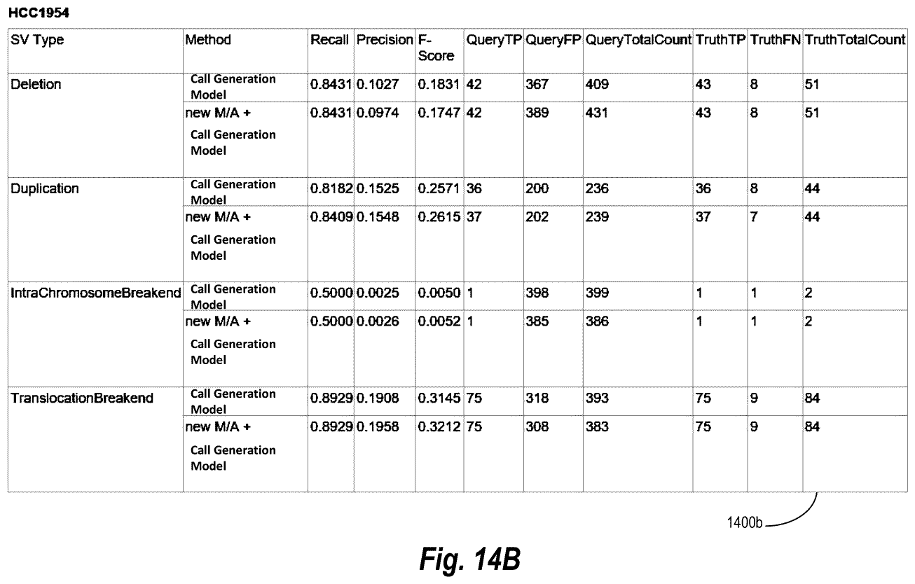

Due in part to improved split-read alignment, the split-read alignment system also improves the accuracy of corresponding nucleobase calls. Based on more accurate split-read alignments, the split-read alignment system can accurately identify and report a split alignment when a read aligns with an alternate-contiguous sequence. The split-read alignment system may report a split alignment in a primary assembly corresponding to the alternate-contiguous sequence to further guide selection a predicted split group. Because of the improved alignment, the split-read alignment system can also determine more accurate variant calls or other nucleobase calls with a higher confidence rate than existing sequencing systems. As illustrated by FIGS. 11A-11D, for example, the split-read alignment system determines more accurate true-negative variant calls for candidate gene fusion events than an existing sequencing system. Additionally, as shown by FIGS. 13 and 14A-14B, the split-read alignment system determines more accurate SNP calls, indel calls, and variant calls than an existing sequencing system.

Beyond the improved alignment and improved base-calling accuracy, in some embodiments, the split-read alignment system improves computational efficiency by reducing the number of sequencing assays and computational devices used to determine structural variant calls. As noted above, some existing sequencing systems consume significant computer processing and time by running both (i) WGS on a specialized sequencing device to generate nucleotide reads for a genomic sample and (ii) multiple genotyping microarrays on a microarray device. By comparing the nucleotide reads to a reference genome for WGS and analyzing light signals from DNA probes in a microarray, existing sequencing systems can determine accurate variant calls for both SNPs and smaller indels based on a reference genome, on the one hand, and targeted structural variants from DNA probes, on the other hand. In contrast to such existing sequencing systems, in some embodiments, the split-read alignment system facilitates a more computationally efficient approach by using a specialized sequencing device to determine nucleotide reads with candidate split groups—without or with fewer genotyping microarrays for targeted structural variants—to determine variant calls corresponding to structural variants or primary-assembly regions of a reference genome. Accordingly, the split-read alignment system can obviate some or all genotyping microarrays for structural variants by determining split group scores for candidate split groups comprising fragment alignments from fragments of a nucleotide read and selecting a predicted split group from among the candidates based on such split group scores.

As illustrated by the foregoing discussion, the present disclosure utilizes a variety of terms to describe the features and advantages of the split-read alignment system. Additional detail is now provided regarding the meaning of such terms. For example, as used herein, the term “nucleotide read” (or simply “read”) refers to an inferred sequence of one or more nucleobases (or nucleobase pairs) from all or part of a sample nucleotide sequence (e.g., a sample genomic sequence, cDNA). In particular, a nucleotide read includes a determined or predicted sequence of nucleobase calls for a nucleotide sequence (or group of monoclonal nucleotide sequences) from a sample library fragment corresponding to a genome sample. For example, in some cases, a sequencing device determines a nucleotide read by generating nucleobase calls for nucleobases passed through a nanopore of a nucleotide-sample slide, determined via fluorescent tagging, or determined from a cluster in a flow cell.

A nucleotide read can include both genomic nucleotide reads based on a DNA sequence and transcriptomic nucleotide reads based on ribonucleic acid (RNA). As used herein, the term “genomic read” refers to a nucleotide read representing an inferred sequence of nucleobases (or nucleobase pairs) derived from genomic DNA (gDNA) extracted from a sample. For example, a genomic read includes a read comprising gDNA that is (i) extracted from or derived from gDNA extracted from a sample and (ii) part of a sample library fragment corresponding to the sample. In some cases, a genomic read includes reads comprising adapter sequences for Assay for Transposase-Accessible Chromatin (ATAC) reads, which are also called ATAC reads. In some embodiments, genomic reads may include, but are not limited to, DNase 1 hypersensitive sites (DNase) sequencing reads, Formaldehyde-Assisted Isolation of Regulatory Elements (FAIRE) sequencing reads, or Tet-Assisted Bisulfite (TAB) sequencing reads.

Conversely, as used herein, the term “transcriptomic read” refers to a nucleotide read representing an inferred sequence of nucleobases (or nucleobase pairs) that either complement or represent RNA extracted from a sample. For example, a transcriptomic read includes a read comprising cDNA that is (i) synthesized from single-stranded messenger RNA (mRNA) or microRNA (miRNA) or derived from RNA extracted from a sample and (ii) part of a sample library fragment corresponding to the sample. As a further example, a transcriptomic read includes a read comprising RNA (e.g., mRNA, miRNA, transfer RNA (tRNA)) that is (i) extracted from or derived from RNA extracted from a sample and (ii) part of a sample library fragment corresponding to the sample.

Additionally, as used herein, the term “genomic coordinate” refers to a particular location or position of a nucleotide base within a genome (e.g., an organism's genome or a reference genome). In some cases, a genomic coordinate includes an identifier for a particular chromosome of a genome and an identifier for a position of a nucleotide base within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1:1234570 or chr1:1234570-1234870). Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleotide-base within the source for the reference genome (e.g., mt:16568 or SARS-CoV-2:29001). By contrast, in certain cases, a genomic coordinate refers to a position of a nucleotide-base within a reference genome without reference to a chromosome or source (e.g., 29727).

As used herein, a “genomic region” refers to a range of genomic coordinates. Like genomic coordinates, in certain implementations, a genomic region may be identified by an identifier for a chromosome and a particular position or positions, such as numbered positions following the identifier for a chromosome (e.g., chr1:1234570-1234870). In various implementations, a genomic coordinate includes a position within a reference genome. In some cases, a genomic coordinate is specific to a particular reference genome.

Also, as used herein, the term “genomic sample” refers to a target genome or portion of a genome undergoing sequencing. For example, a sample genome includes a sequence of nucleotides isolated or extracted from a sample organism (or a copy of such an isolated or extracted sequence). In particular, a sample genome includes a full genome that is isolated or extracted (in whole or in part) from a sample organism and composed of nitrogenous heterocyclic bases. A sample genome can include a segment of deoxyribonucleic acid (DNA), ribonucleic acid (RNA), or other polymeric forms of nucleic acids or chimeric or hybrid forms of nucleic acids noted below. In some cases, the sample genome is found in a sample prepared or isolated by a kit and received by a sequencing device.

As used herein, the term “split group” refers to a group of one or more fragment alignments corresponding to a nucleotide read. In particular, a split group comprises a chain of one or more fragment alignments forming a split-alignment of one nucleotide read with respect to a reference genome. For example, a split group may comprise fragment alignments of one or more fragments of a nucleotide read. Such fragment alignments can represent alignments of read fragments from a single-end nucleotide read or a paired-end nucleotide read (e.g., a mate) from a pair of paired-end nucleotide reads. Relatedly, the term “candidate split group” refers to potential fragment alignments of one nucleotide read.

Further, the term “predicted split group” refers to a selected split group to represent an alignment of a nucleotide read. In particular, a predicted split group includes a split group having a highest split group score from among candidate split groups corresponding to a nucleotide read. In some embodiments, a predicted split group accordingly represents a prediction that the corresponding split alignment most likely represents a true alignment of the nucleotide read with a reference genome. For example, in certain circumstances described below, the predicted split group may represent a split read alignment corresponding to a true structural variant in the sequenced genomic sample.

As used herein, the term “split group score” refers to a numeric score, metric, or other quantitative measurement indicating an accuracy of fragment alignments in a split group. For instance, a split group score indicates the likelihood that a given split alignment of one or more fragment alignments of a candidate split group is correct with respect to a reference genome. For example, as explained below, a split group score may reflect a combination of fragment alignment scores, a break penalty, an overlap penalty, and, in some cases, a gap penalty for fragment alignments within a split group.

As used herein, the term “fragment alignment” refers to a candidate local alignment of a given fragment of a nucleotide read with respect to a reference genome. For example, a fragment alignment indicates a genomic region or genomic coordinates of a reference genome with which a fragment of a read aligns.

As further used herein, the term “alignment score” refers to a numeric score, metric, or other quantitative measurement evaluating an accuracy of an alignment between a nucleotide read or a fragment of the nucleotide read and another nucleotide sequence from a reference genome. In particular, an alignment score includes a metric indicating a degree to which the nucleobases of a nucleotide read (or fragment of the nucleotide read) match or are similar to a reference sequence or an alternate contiguous sequence from a reference genome. In certain implementations, an alignment score takes the form of a Smith-Waterman score or a variation or version of a Smith-Waterman score for local alignment, such as various settings or configurations used by DRAGEN by Illumina, Inc. for Smith-Waterman scoring. Accordingly, the term “fragment alignment score” refers to an alignment score for a fragment alignment of a nucleotide read. Accordingly, in a split group comprising multiple fragment alignments, a fragment alignment score may be determined for each fragment alignment within the split group.

Relatedly, the term “alternate contiguous sequence” (or simply “alt contig”) refers to a contiguous sequence representing a population haplotype added to a linear reference genome (or other reference genome) at a particular genomic coordinate or genomic coordinates (e.g., lifted over to the linear reference genome). In some implementations, a graph reference genome can include alternate contiguous sequences mapped to genomic coordinates of a primary assembly for a linear reference genome. For example, an alternate contiguous sequence may represent a population haplotype containing a structural variant with liftover to two or more genomic coordinates in the linear reference genome corresponding to two or more flanks of structural variant breakends. In some cases, a hash table for a graph reference genome includes identifiers that associate alternate contiguous sequences representing structural variant haplotypes with genomic coordinates representing reference haplotypes from a primary assembly for a linear reference genome.

Relatedly, the term “alt-contig fragment alignment score” refers to an alignment score for an alignment between one or more read fragments with an alternate contiguous sequence. In particular, an alt-contig fragment alignment score can include an alignment score for an alignment of one or more inner read fragments and one or more outer read fragments of a nucleotide read with an alternate contiguous sequence. As explained below, an alt-contig fragment alignment score may replace or serve as a split group score under certain circumstances.

As further used herein, the term “break penalty” refers to a numeric score, metric, or other quantitative measurement penalizing fragment alignments within a split group that exhibit a break between or among the fragment alignments. In particular, a break penalty can include a metric that penalizes fragment alignments of a split group to a degree (or in proportion to) the fragment alignments exhibit a break of nucleobases between the fragment alignments at a breakpoint. Accordingly, in some embodiments, the split-read alignment system determines relatively higher break penalties for breaks between or among fragment alignments of relatively larger size or distance.

Relatedly, the term “breakpoint” refers to a break or space between nucleotide reads and/or fragments of nucleotide reads where nucleotide reads align with different locations within a reference genome. For example, a split alignment contains a breakpoint because the fragments of the nucleotide read exhibit highest scoring alignments (e.g., highest pair scores) with a reference genome when they align to different locations that have a break or breakpoint between the fragments of the nucleotide read.

As further used herein, the term “overlap penalty” refers to a numeric score, metric, or other quantitative measurement penalizing fragment alignments within a split group that overlap within a nucleotide read. In particular, an overlap penalty can include a metric that penalizes fragment alignments of a split group to a degree (or in proportion to) the fragment alignments exhibit overlapping nucleotide bases within a nucleotide read. For example, a 150-base-pair nucleotide read may have at least two fragment alignments. The first fragment alignment may align with the leftmost 100 base pairs to one chromosome within a reference genome (e.g., Chr1), and the second fragment alignment may align with the rightmost 100 base pairs to another chromosome (e.g., Chr2). Despite the example fragment alignments not overlapping within the reference genome, the first and second fragment alignments may nevertheless overlap by 50 base pairs within the nucleotide read. An overlap penalty can accordingly represent a metric penalizing such a 50-base-pair overlap within the nucleotide read from the foregoing example (or other example overlap of nucleotide bases).

As further used herein, the term “gap penalty” refers to a numeric score, metric, or other quantitative measurement penalizing a pair of fragment alignments based on a gap between the pair of fragment alignments within a nucleotide read. In particular, the gap penalty can include a metric that penalizes fragment alignments of a split group to a degree (or in proportion to) the size of a gap existing between the fragment alignments within a nucleotide read. For example, a 150-base-pair nucleotide read may have at least two fragment alignments. The first fragment alignment may align the leftmost 50 base pairs to a first set of genomic coordinates of a reference genome, and the second fragment alignment may align the rightmost 50 base pairs to a second set of genomic coordinates of the reference genome. In contrast to the overlap example above, the nucleotide read may include a 50 base-pair gap within the nucleotide read in between a first fragment corresponding to the first fragment alignment and a second fragment corresponding to the second fragment alignment. A gap penalty can accordingly represent a metric penalizing such a 50 base-pair gap between the first fragment alignment and the second fragment alignment within the nucleotide read.

As used herein, the term “split alignment” refers to an alignment of different fragments of a read to different regions in a reference genome. For example, a split alignment can refer to a split-read or chimeric alignment.

As further used herein, the term “pair score” refers to a numeric score, metric, or other quantitative measurement evaluating an accuracy of alignments between a candidate pair of split groups and nucleotide sequences from a reference genome. In particular, a pair score includes a metric indicating a degree to which a candidate pair of split groups is accurately aligned with a nucleotide sequence from a reference genome. More specifically, in some embodiments, a pair score indicates a likelihood that a candidate pair of split groups comprise true mates of a paired-end nucleotide read. Indeed, in some embodiments, a pair score represents a sum of split group scores for respective candidate pairs of split groups minus a pairing penalty.

As used herein, the term “pairing penalty” refers to a numeric score, metric, or other quantitative measurement penalizing a pair of fragment alignments that are unlikely mates of a paired-end read. In particular, the term pairing penalty refers to a metric indicating a likelihood or unlikelihood of fragment alignments being correctly paired based on a geometry of two or more fragment alignments with respect to a reference genome. For example, the pairing penalty can represent a log likelihood or, alternatively, a log P-value of an insert size between two innermost fragment alignments based on an empirical insert distribution.

As used herein, the term “reference genome” refers to a digital nucleic acid sequence assembled as a representative example (or representative examples) of genes and other genetic sequences of an organism. Regardless of the sequence length, in some cases, a reference genome represents an example set of genes or a set of nucleic acid sequences in a digital nucleic acid sequence determined as representative of an organism. For example, a linear human reference genome may be GRCh38 (or other versions of reference genomes) from the Genome Reference Consortium. While GRCh38 may include alternate contiguous sequences representing alternate haplotypes, such as SNPs and small indels (e.g., 10 or fewer base pairs, 50 or fewer base pairs), GRCh38 includes alternate haplotypes with limited representation of population structural variants. Indeed, the structural variants represented in GRCh38 include only those represented by the 11 individuals whose libraries GRCh38 is constructed upon. Relatedly, the term “reference region” refers to a portion or a fraction of a reference genome. For example, a reference region may be a selected number of nucleobases (e.g., 150 bases) from the reference genome.

As used herein, the term “variant” refers to a nucleobase or multiple nucleobases that do not align with, differ from, or vary from a corresponding nucleobase (or nucleotide bases) in a reference sequence or a reference genome. For example, a variant includes an SNP, an indel, or a structural variant that indicates nucleobases in a sample nucleotide sequence that differ from reference nucleobases in corresponding genomic coordinates of a reference sequence. Similarly, a “variant-nucleobase call” refers to a nucleobase call comprising a variant at a particular genomic coordinate. Conversely, a “non-variant-nucleobase call” refers to a nucleobase call comprising a non-variant (or matching a reference base) at a genomic coordinate.

Additionally, as used herein, the term “nucleobase call” (or sometimes simply “nucleotide-base call” or “base call”) refers to a determination or prediction of a particular nucleobase (or nucleobase pair) for an oligonucleotide (e.g., nucleotide read) during a sequencing cycle or for a genomic coordinate of a sample genome. In particular, a nucleobase call can indicate (i) a determination or prediction of the type of nucleobase that has been incorporated within an oligonucleotide on a nucleotide-sample slide (e.g., read-based nucleobase calls) or (ii) a determination or prediction of the type of nucleobase that is present at a genomic coordinate or region within a genome, including a variant call or a non-variant call in a digital output file. In some cases, for a nucleotide read, a nucleobase call includes a determination or a prediction of a nucleobase based on intensity values resulting from fluorescent-tagged nucleotides added to an oligonucleotide of a nucleotide-sample slide (e.g., in a cluster of a flow cell). Alternatively, a nucleobase call includes a determination or a prediction of a nucleobase from chromatogram peaks or electrical current changes resulting from nucleotides passing through a nanopore of a nucleotide-sample slide. By contrast, a nucleobase call can also include a final prediction of a nucleobase at a genomic coordinate of a sample genome for a variant call file (VCF) or other base-call-output file-based on nucleotide reads corresponding to the genomic coordinate. Accordingly, a nucleobase call can include a base call corresponding to a genomic coordinate and a reference genome, such as an indication of a variant or a non-variant at a particular location corresponding to the reference genome. Indeed, a nucleobase call can refer to a variant call, including but not limited to, a single nucleotide variant (SNV), an insertion or a deletion (indel), or base call that is part of a structural variant. As suggested above, a single nucleobase call can be an adenine (A) call, a cytosine (C) call, a guanine (G) call, a thymine (T) call, or a uracil (U) call.

As further used herein, the term “alignment file” refers to a digital file that indicates the relative alignment or mapping of nucleotide reads with nucleotide sequences of a reference genome or other reference nucleotide sequences. In particular, an alignment file can include data indicating relative mapping position of nucleotide reads and nucleotide sequences of a reference genome. In some embodiments, an alignment file includes or constitutes a Sequence Alignment/Map (SAM) file, a Binary Alignment Map (BAM) file, a FAST-All (FASTA) file, or a FASTQ file.

As used herein, the term “variant call file” refers to a digital file that indicates or represents one or more nucleobase calls (e.g., variant calls) compared to a reference genome along with other information about the nucleobase calls (e.g., variant calls). For example, a variant call format (VCF) file refers to a text file format that contains information about variants at specific genomic coordinates, including meta-information lines, a header line, and data lines where each data line contains information about a single nucleobase call (e.g., a single variant).

In some embodiments, the split-read alignment system or a corresponding sequencing system utilizes a call generation model to determine nucleotide-base calls (e.g., variant calls or genotype calls). As used herein, the term “call generation model” refers to a probabilistic model that generates sequencing data from nucleotide reads of a sample nucleotide sequence, including nucleobase calls, variant calls, and/or genotype calls along with associated metrics. Accordingly, in some cases, a call generation model may be a variant call generation model. For example, in some cases, a call generation model refers to a Bayesian probability model that generates variant calls based on nucleotide reads of a sample nucleotide sequence. Such a model can process or analyze sequencing metrics corresponding to read pileups (e.g., multiple nucleotide reads corresponding to a single genomic coordinate), including mapping quality, base quality, and various hypotheses including foreign reads, missing reads, joint detection, and more. A call generation model may likewise include multiple components, including, but not limited to, different software applications or components for mapping and aligning, sorting, duplicate marking, computing read pileup depths, and variant calling. In some cases, a call generation model refers to an ILLUMINA DRAGEN model for variant calling functions and mapping and alignment functions (e.g., a DRAGEN variant caller or “DRAGEN VC”).

As used herein, for example, the term “configurable processor” refers to a circuit or chip that can be configured or customized to perform a specific application. For instance, a configurable processor includes an integrated circuit chip that is designed to be configured or customized on site by an end user's computing device to perform a specific application. Configurable processors include, but are not limited to, an ASIC, ASSP, a coarse-grained reconfigurable array (CGRA), or FPGA. By contrast, configurable processors do not include a CPU or GPU. In some embodiments, the split-read alignment system uses a configurable processor (e.g., FPGA) or a processor (e.g., CPU) to perform the various embodiments described herein.

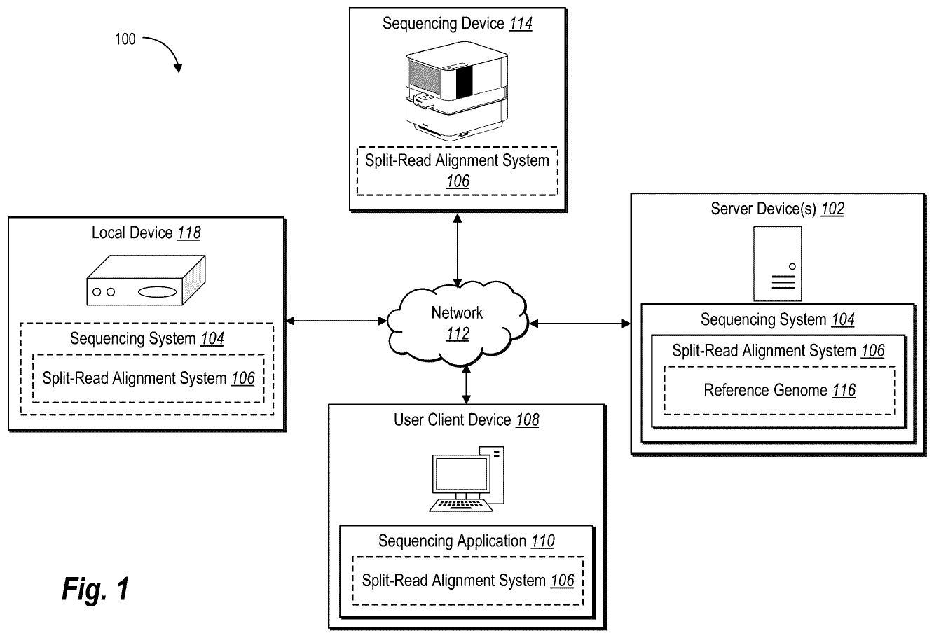

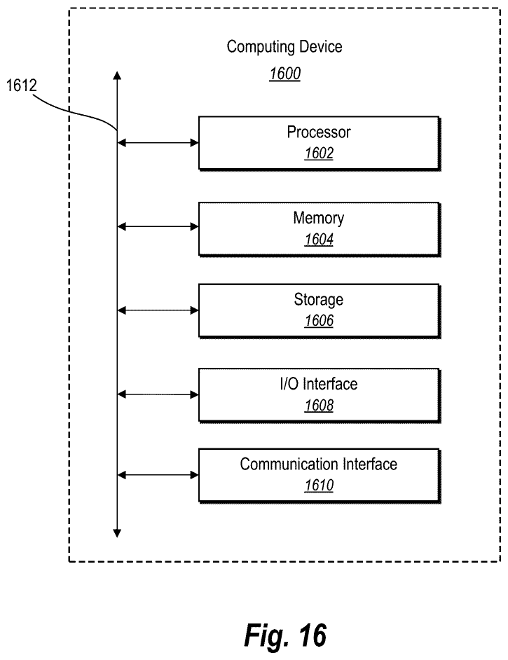

The following paragraphs describe the split-read alignment system with respect to illustrative figures that portray example implementations and embodiments. For example, FIG. 1 illustrates a schematic diagram of a computing system 100 in which a split-read alignment system 106 operates in accordance with one or more implementations. As illustrated, the computing system 100 includes one or more server device(s) 102 connected to a user client device 108, a local device 118, and a sequencing device 114 via a network 112. The network 112 can comprise any suitable network over which computing devices can communicate. Example networks are discussed in additional detail below with respect to FIG. 16.

As shown in FIG. 1, the computing system 100 includes the server device(s) 102. In various implementations, the server device(s) 102 may generate, receive, analyze, store, and transmit digital data, such as data for nucleobase calls or sequenced nucleic-acid polymers. In some implementations, the server device(s) 102 receive various data from the sequencing device 114, such as data from a sample genome and/or nucleotide reads. The server device(s) 102 may also communicate with the user client device 108. In particular, the server device(s) 102 can send data for nucleotide reads, direct nucleobase calls, nucleobase calls, and/or sequencing metrics to the user client device 108.

As shown, the server device(s) 102 includes a sequencing system 104. In general, the sequencing system 104 analyzes the data (e.g., call data) received from the sequencing device 114 or elsewhere to determine nucleobase sequences for nucleic-acid polymers. For example, the sequencing system 104 can receive raw data from the sequencing device 114 and determine a nucleobase sequence for a sample genome or a nucleic-acid segment. In some implementations, the sequencing system 104 determines the sequences of nucleobases in DNA and/or RNA segments or oligonucleotides.

As also shown, the sequencing system 104 includes the split-read alignment system 106. As described below, the split-read alignment system 106 can determine split-read alignments of nucleotide reads with a reference genome 116. For example, in some embodiments, the split-read alignment system 106 identifies one or more nucleotide reads corresponding to a genomic region of a genomic sample. The split-read alignment system 106 further (i) determines candidate split groups comprising fragment alignments corresponding to the one or more nucleotide reads and (ii) generates split group scores for split alignments of the candidate split groups with the reference genome 116. Based on the split group scores, the split-read alignment system 106 selects a predicted split group from among the candidate split groups to use for nucleobase calling.

As further shown in FIG. 1, the computing system 100 includes the user client device 108. In various implementations, the user client device 108 can generate, store, receive, and send digital data. In particular, the user client device 108 can receive the data from the sequencing device 114. As further illustrated, the user client device 108 includes a sequencing application 110. The sequencing application 110 may be a web application or a native application stored and executed on the user client device 108 (e.g., a mobile application, desktop application, or web application). The sequencing application 110 can receive data from the sequencing system 104 and/or split-read alignment system 106. For example, the user client device 108 can receive variant call files and/or alignment files from the sequencing system 104.

The sequencing application 110 can also include instructions that (when executed) cause the user client device 108 to receive data from the split-read alignment system 106 and present data from the sequencing device 114 and/or the server device(s) 102. Furthermore, the sequencing application 110 can instruct the user client device 108 to display data for nucleobase calls with respect to the reference genome 116, such as nucleobase calls or an indication of a split alignment from a variant call file or an alignment file. Indeed, the user client device 108 can display nucleobase call results for a genome sample and/or an indication of a predicted split group.

As further shown in FIG. 1, the computing system 100 optionally includes the sequencing device 114. In various implementations, the sequencing device 114 can sequence a genomic sample or other nucleic-acid polymer. For example, the sequencing device 114 analyzes nucleic-acid segments or oligonucleotides extracted from genomic samples to generate data either directly or indirectly on the sequencing device 114. More particularly, the sequencing device 114 receives and analyzes, within nucleotide-sample slides (e.g., flow cells), nucleic-acid sequences extracted from genomic samples. In one or more implementations, the sequencing device 114 utilizes SBS to sequence a genomic sample or other nucleic-acid polymers. In addition to, or in the alternative to communicating across the network 112, in some implementations, the sequencing device 114 bypasses the network 112 and communicates directly with the user client device 108.

As further depicted in FIG. 1, in some implementations, the server device(s) 102 includes a distributed collection of servers, where the server device(s) 102 include several server devices distributed across the network 112 and located in the same or different physical locations. For instance, the server device(s) 102 can be implemented, in whole or in part, on the local device 118. To illustrate, the local device 118 may implement the sequencing system 104 and/or the split-read alignment system 106. Further, the server device(s) 102 and/or the local device 118 can include a content server, an application server, a communication server, a web-hosting server, or another type of server.

The user client device 108 illustrated in FIG. 1 can include various types of client devices. For example, in some implementations, the user client device 108 includes non-mobile devices, such as desktop computers or servers, or other types of client devices. In various implementations, the user client device 108 includes mobile devices, such as laptops, tablets, mobile telephones, or smartphones. Additional details with regard to the user client device 108 are discussed below with respect to FIG. 16.

Moreover, while the split-read alignment system 106 is shown on the server device(s) 102, as part of the sequencing system 104, in some implementations, the split-read alignment system 106 is implemented by (e.g., located entirely or in part) on the user client device 108, the sequencing device 114, and/or the local device 118. As mentioned, in some implementations, the split-read alignment system 106 is implemented by one or more other components of the computing system 100, such as the sequencing device 114. In particular, the split-read alignment system 106 can be implemented in a variety of different ways across the server device(s) 102, the network 112, the user client device 108, the local device 118, and the sequencing device 114.

Though FIG. 1 illustrates the components of the computing system 100 communicating via the network 112, in certain implementations, the components of computing system 100 can also communicate directly with each other, bypassing the network 112. For instance, in some implementations, the user client device 108 communicates directly with the sequencing device 114. Additionally, in some implementations, the user client device 108 communicates directly with the split-read alignment system 106 and/or the server device(s) 102. In some implementations, the user client device 108 communicates directly with the local device 118. Moreover, the split-read alignment system 106 can access one or more databases housed on or accessed by the server device(s) 102 or elsewhere in the computing system 100.

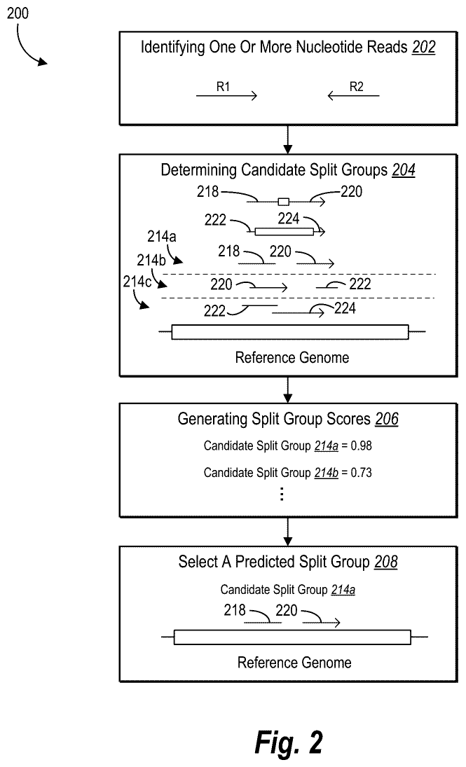

FIG. 2 provides an overview of the split-read alignment system 106 determining scores for alignments of one or more fragments from a nucleotide read in a candidate split group and selecting a predicted split group from among the candidates based on such scores to use for base calling in accordance with one or more embodiments. Generally, and as illustrated in FIG. 2, the split-read alignment system 106 performs a series of acts 200 including an act 202 of identifying one or more nucleotide reads. The split-read alignment system 106 further performs an act 204 of determining candidate split groups comprising fragment alignments for fragments of the nucleotide reads. The split-read alignment system 106 also performs an act 206 of generating split group scores for the determined candidate split groups and an act 208 of selecting a predicted split group.

As illustrated in FIG. 2, the split-read alignment system 106 performs the act 202 of identifying one or more nucleotide reads. In particular, the split-read alignment system 106 identifies one or more nucleotide reads corresponding to a genomic region of a genomic sample. For example, the split-read alignment system 106 may identify nucleotide reads corresponding to a template strand or sequence of a genomic sample. More specifically, a template comprises an original contiguous DNA or RNA fragment sequenced by either single-end or paired-end methods. In the single-end method, a single read is sequenced from one end of the template. Because the single-end read is sequenced from one end of the template, the single read represents the complimentary sequence of a template. In the paired-end method, a first read (e.g., R1) is sequenced from one end of the template toward the middle and a second read (e.g., R2) is sequenced from the other end. FIG. 2 illustrates two paired-end reads R1 and R2 oriented toward each other. As illustrated, there is a gap between R1 and R2, however, overlap between R1 and R2 is also possible. R1 and R2 may be described as paired-end mates.

As further illustrated in FIG. 2, the split-read alignment system 106 performs the act 204 of determining candidate split groups. In particular, the split-read alignment system 106 determines candidate split groups comprising fragment alignments corresponding to the one or more nucleotide reads. Generally, fragment alignments refer to candidate local alignments of fragments of a read. FIG. 2 illustrates the split-read alignment system 106 determining candidate split groups for R1. R1 may be a single-end read or one of two paired-end reads. R1 may comprise different one or more fragments.

To illustrate fragments and fragment alignments, FIG. 2 shows the split-read alignment system 106 identifying various fragments of a nucleotide read. As illustrated, the split-read alignment system 106 identifies a fragment 218, a fragment 220, a fragment 222, and a fragment 224 corresponding to R1. The fragments illustrated in FIG. 2 are separated by breaks representing structural variant (or “SV”) breakpoints. While FIG. 2 illustrates R1 broken by a single SV breakpoint, a nucleotide read may have no SV breakpoints or several SV breakpoints. For example, the fragment 220 may be further broken into two or more fragments.

As further illustrated in FIG. 2, the split-read alignment system 106 determines candidate split groups 214a-214c for R1. As part of performing the act 204, the split-read alignment system 106 identifies fragment alignments for the identified fragments of the read. Generally, fragments of a read may be aligned with different sequences in a reference genome. For instance, the fragments 218 and 220 may be aligned with nearby genomic regions of the reference genome on a same chromosome. Conversely, the fragment 218 may be aligned with the reference genome at one chromosome and the fragment 220 may be aligned with the reference genome at another chromosome.

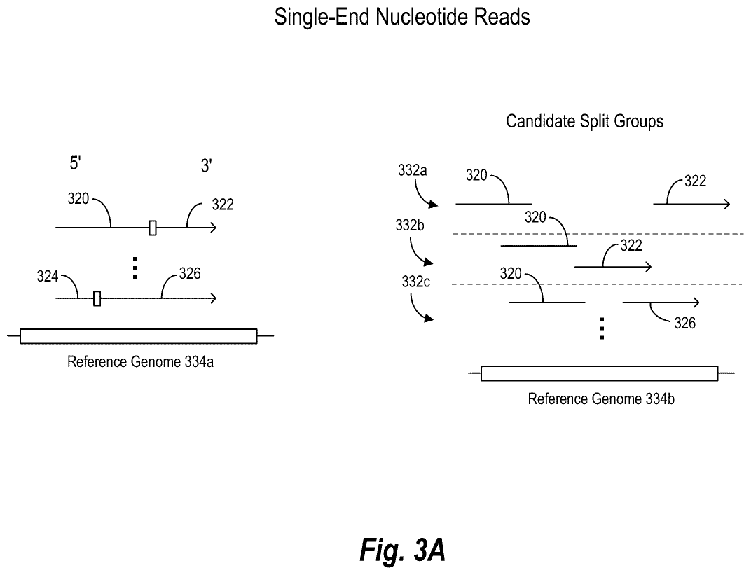

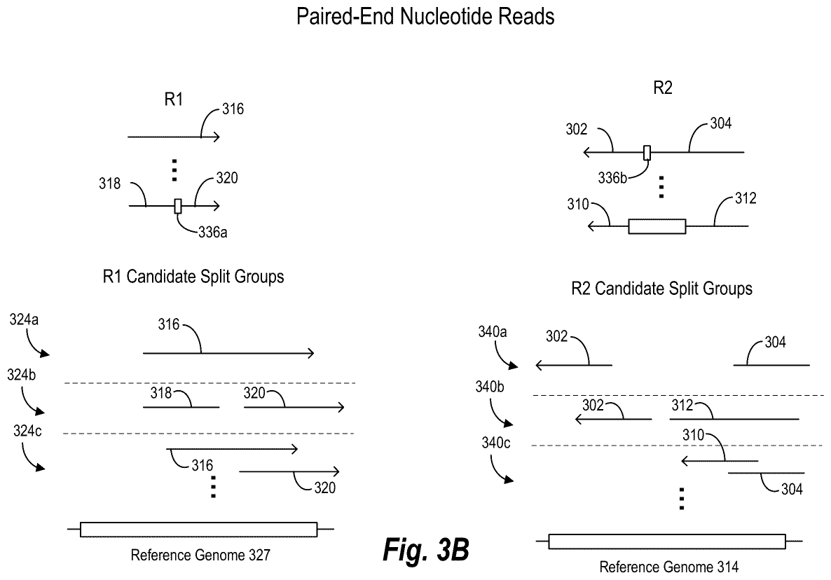

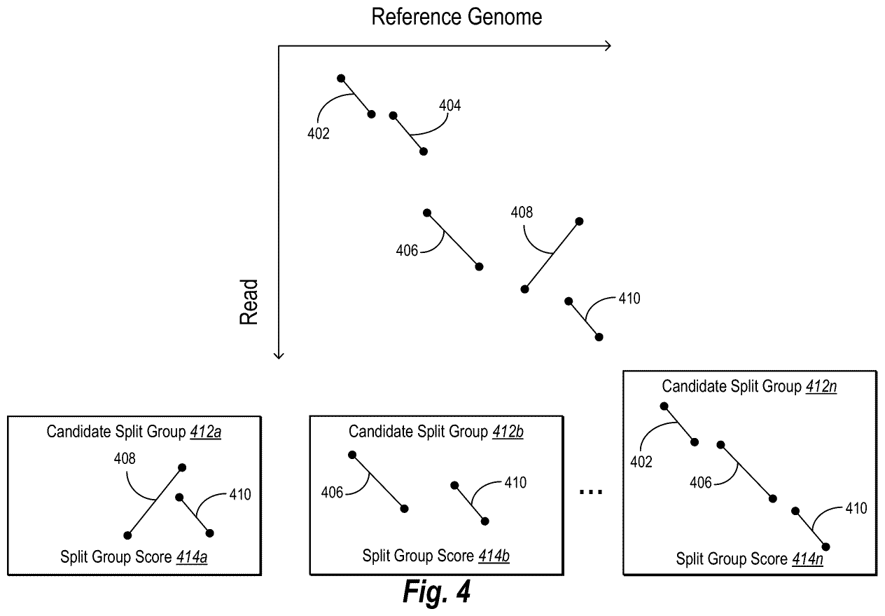

FIG. 2 illustrates candidate fragment alignments corresponding to R1 as part of split groups. More specifically, the candidate split groups 214a-214c show candidate local alignments of different combinations of the fragments 218-222 of R1 on a reference genome. For instance, the candidate split group 214a comprises candidate fragment alignments of the fragment 218 and the fragment 220 relative to the reference genome. FIGS. 3A-4 illustrate and the corresponding paragraphs further detail the split-read alignment system 106 determining candidate split groups for single-end and paired-end nucleotide reads in accordance with one or more embodiments.

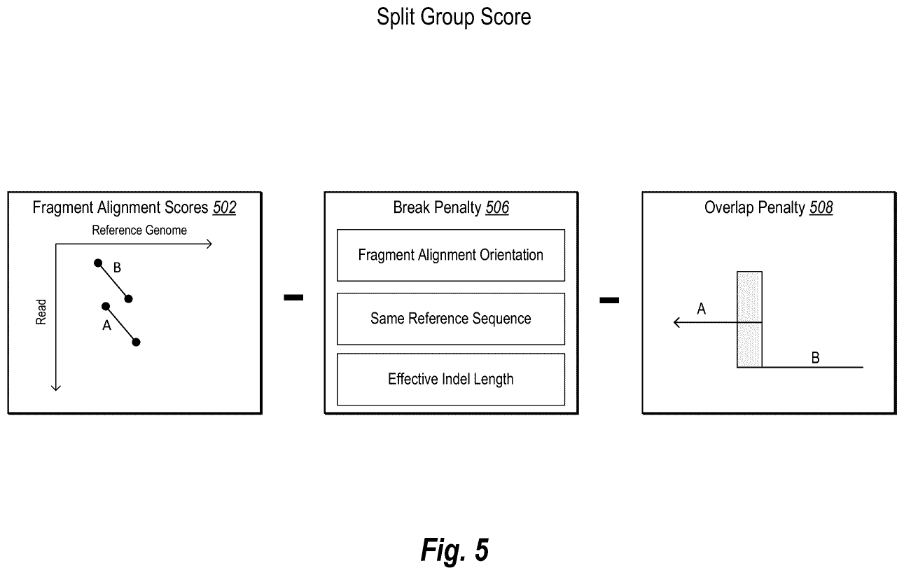

As further illustrated in FIG. 2, the split-read alignment system 106 performs the act 206 of generating split group scores. Generally, the split-read alignment system 106 generates split group scores for split alignments of the candidate split groups with a reference genome. The split-read alignment system 106 can generate a split group score for a split group based on fragment alignment scores, a break penalty, and an overlap penalty. As illustrated, the split-read alignment system 106 generates a split group score of 0.98 for the candidate split group 214a and a split group score of 0.73 for the candidate split group 214b. FIG. 5 illustrates and the corresponding discussion provide additional details regarding determining a split group score in accordance with one or more embodiments.

After determining split group scores, as further shown in FIG. 2, the split-read alignment system 106 performs the act 208 of selecting a predicted split group. The split-read alignment system 106 selects a predicted split group from the candidate split groups based on the split group scores. To illustrate, in some embodiments, the split-read alignment system 106 selects the candidate split group 214a as a predicted split group based on the candidate split group 214a having the highest split group score.

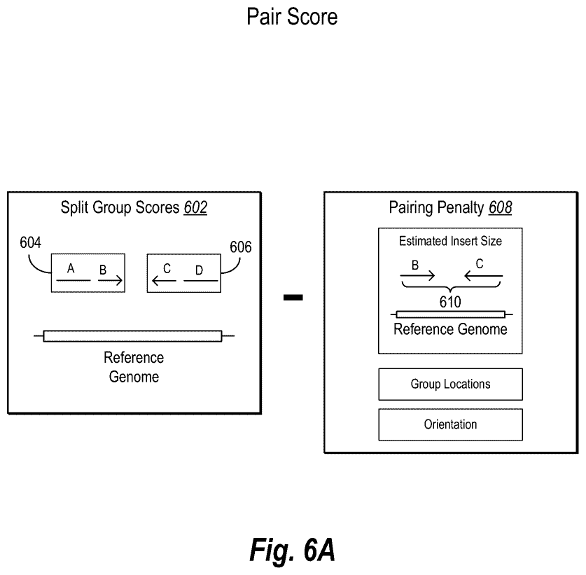

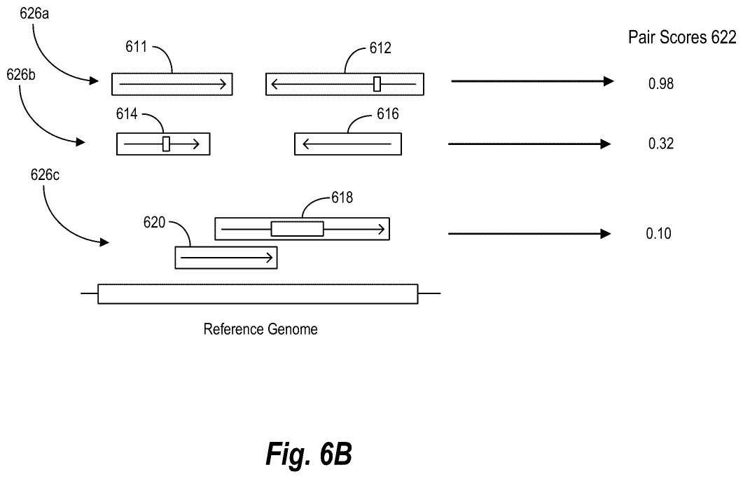

As mentioned, the split-read alignment system 106 may generate predicted split groups for single-end and paired-end reads. In some implementations, the split-read alignment system 106 predicts a split group based, in part, on pair scores for pairs of candidate split groups. FIGS. 6A-6B illustrate the split-read alignment system 106 generating a pair score in accordance with one or more embodiments.

As mentioned previously, the split-read alignment system 106 determines candidate split groups for single-end nucleotide reads and paired-end nucleotide reads. FIG. 3A illustrates the split-read alignment system 106 determining candidate split groups for a single-end nucleotide read, and FIG. 3B illustrates the split-read alignment system 106 determining candidate split groups for paired-end nucleotide reads in accordance with one or more embodiments.

FIG. 3A illustrates the split-read alignment system 106 identifying candidate split groups in single-end nucleotide reads. As mentioned previously, single-end read sequencing involves sequencing DNA or RNA from one direction. Generally, the split-read alignment system 106 identifies fragments of a nucleotide read. To illustrate, the split-read alignment system 106 identifies a fragment 320, a fragment 322, a fragment 324, and a fragment 326. The illustrated fragments are divided by potential breakpoints when aligned with a reference genome 334a.

The split-read alignment system 106 identifies candidate split groups 332a-332c of the identified fragments. Generally, the candidate split groups 332a-332c comprise all realistic fragment alignments. In other words, the candidate split groups 332a-332c include potential fragment alignments for read fragments with a reference genome 334b. For instance, the candidate split group 332a includes fragment alignments for the fragment 320 and the fragment 322 with respect to the reference genome 334b. The candidate split group 332b includes overlapping fragment alignments of the fragment 320 and the fragment 322. The candidate split group 332c includes fragment alignments of the fragment 320 and the fragment 326 with respect to the reference genome 334b.

While FIG. 3A illustrates the candidate split groups 332a-332c, additional candidate split groups are possible. For instance, a candidate split group can comprise a single fragment alignment of a single fragment of the nucleotide read. For instance, in some embodiments, the fragment may be the entire nucleotide read. Furthermore, the candidate split groups can comprise more than two fragment alignments. For instance, a candidate split group can comprise fragment alignments for three or more fragments of a nucleotide read.

As mentioned, FIG. 3B illustrates the split-read alignment system 106 determining candidate split groups for nucleotide reads in paired-end sequencing in accordance with one or more embodiments. Generally, paired-end sequencing sequences includes generating paired nucleotide reads that begin at different (and opposite) positions of a library template. In particular, paired-end sequencing generates two mate reads. For instance, R1 and R2 illustrated in FIG. 3B comprise paired mates. As mentioned, a gap may exist between R1 and R2 or the paired-end reads may overlap.

In some instances, one paired-end mate crosses a breakpoint (e.g., an SV breakpoint) while the other paired-end mate does not. To illustrate, R2 may cross a breakpoint while R1 does not. Accordingly, R2 may be segmented into a fragment 302 and a fragment 304, while R1 remains a whole fragment 316. In this example, the 3′ end of R2 (e.g., inner end of the fragment 302) is in a properly paired position relative to the mate alignment of the whole fragment 316 while the fragment 304 may be potentially aligned at a different genomic region a reference genome.

In another example, R1 and R2 may overlap and both cross a single breakpoint. To illustrate, break 336a and break 336b can represent the same breakpoint. In this example, a fragment 318 of R1 overlaps with a fragment 302 of R2, and a fragment 320 of R1 represents with a fragment 304 of R2.

In another example, R1 and R2 cross different breakpoints. For example, the break 336a can represent a different breakpoint than a break 336b. Thus, R1 is split into a fragment 318 and a fragment 320, while R2 is split into a fragment 310 and a fragment 312.

The split-read alignment system 106 contemplates the above scenarios by generating candidate split groups for both R1 and R2. As illustrated in FIG. 3B, the split-read alignment system 106 generates candidate split groups 324a-324c corresponding to R1 relative to a reference genome 327. The split-read alignment system 106 also generates candidate split groups 340a, 340b, and 340c corresponding to R2 relative to a reference genome 314. In some implementations, the reference genome 327 and the reference genome 314 represent the same reference genome. The candidate split groups 324a-324c and the candidate split groups 340a-340c comprise fragment alignments corresponding to a relevant nucleotide read, that is, either R1 or R2.

As mentioned previously with respect to FIG. 3A, in some embodiments, a candidate split group comprises a chain of fragment alignments for one nucleotide read. As noted above, the nucleotide read and fragment alignments can be of various nucleobase lengths. As illustrated, the split-read alignment system 106 can determine that a candidate split group comprises an alignment of whole nucleotide read. For example, the candidate split group 324a comprises an alignment of the whole fragment 316 comprising the whole R1 with respect to the reference genome 327. By contrast, candidate split groups can also comprise overlapping fragment alignments. For example the candidate split group 324c for R1 and the candidate split group 340c for R2 comprise overlapping fragment alignments. The split-read alignment system 106 can further determine candidate split groups that do not overlap. For example, the candidate split group 324b for R1 and the candidate split group 340a for R2 comprise fragment alignments that do not overlap. Furthermore, the split-read alignment system 106 can generate candidate fragments comprising chains of more than two fragment alignments. Furthermore, the candidate fragments can also comprise fragment alignments having different geometric orientations with respect to a reference genome.

FIGS. 3A-3B illustrate the split-read alignment system 106 generating candidate split groups for single-end and paired-end nucleotide reads. In some implementations, the split-read alignment system 106 utilizes dynamic programming to efficiently generate and evaluate all possible fragment alignment sequences. FIG. 4 illustrates and the corresponding discussion describe the split-read alignment system 106 utilizing dynamic programming to generate and evaluate candidate split groups in accordance with one or more embodiments.

By utilizing dynamic programming, in some embodiments, the split-read alignment system 106 considers a subset of every possible candidate split group. More specifically, the split-read alignment system 106 identifies a subset of likely candidate split groups by evaluating fragment alignments in a particular order. To illustrate, in some implementations, the split-read alignment system 106 determines candidate split groups by iteratively grouping individual fragment alignments following an order of outermost fragment alignments to innermost fragment alignments of a nucleotide read. The split-read alignment system 106 further iteratively scores groupings of individual fragment alignments following the order in which the individual fragment alignments were grouped.

Generally, each read has two ends, a 3′ or 5′ end, where the 3′ is designated as “inner” and the 5′ end is designated as “outer.” For paired-end reads, the terms inner and outer refer to expected relative positions in the template. For single-end or paired-end reads with a forward-reverse (FR) pair orientation, the 3′ end represents the inner end, and the 5′ end represents the outer end. When a reverse-forward (RF) or forward-forward (FF)/reverse-reverse (RR) pair orientation is expected, the split-read alignment system 106 determines inner and outer read ends dynamically. In particular, the split-read alignment system 106 designates innermost fragment alignments and outermost fragment alignments according to the observed geometry of the proper pair of fragment alignments with the highest sum of alignment scores.

FIG. 4 illustrates the process of dynamic programming performed by the split-read alignment system 106. As shown for illustrative purposes, fragment alignments 402-410 are organized in a Smith-Waterman matrix. In addition to depicting locations of fragment alignments with respect to a nucleotide read and a reference genome, the Smith-Waterman matrix shows orientations of the fragment alignments 402-410. For example, as depicted, the fragment alignment 406 represents a forward alignment while the fragment alignment 408 represents a reverse-complemented alignment. FIG. 4 depicts the fragment alignments 402-410 as perfect gapless diagonal alignments, but individual fragment alignments of the fragment alignments 402-410 may contain indels (insertions and/or deletions). In some embodiments, such indels are relatively smaller variants in size (e.g., <50 base pairs) as opposed to the size of a structural variant (e.g., >50 base pairs). Small indels are typically aligned within fragment alignments while structural variants are typically described or depicted by multi-fragment split-read alignment.

As illustrated in FIG. 4, the fragment alignment 402 represents an innermost fragment alignment and the fragment alignment 410 represents an outermost fragment alignment. As illustrated, the split-read alignment system 106 begins by grouping an outermost fragment alignment with the next-outermost fragment alignments. For example, the split-read alignment system 106 groups the fragment alignment 410 with a fragment alignment 408. The grouping of the fragment alignment 410 and the fragment alignment 408 make up a candidate split group 412a.

After grouping (and determining a split group score for) the outermost fragment alignment and the next-outermost fragment alignment, the split-read alignment system 106 groups the (and determines a split group score for) the outermost fragment alignment and the next-next-outermost fragment alignment. Accordingly, the split-read alignment system 106 groups the fragment alignment 410 with a fragment alignment 406. The grouping of the fragment alignment 410 and the fragment alignment 406 make up a candidate split group 412b.

In some implementations, as just indicated, the split-read alignment system 106 generates split group scores by iteratively scoring groupings of individual fragment alignments following the order in which the individual fragment alignments were grouped. As illustrated in FIG. 4, the split-read alignment system 106 scores the candidate split group 412a and the candidate split group 412b in the order that they were formed. For instance, the split-read alignment system 106 determines a split group score 414a for the candidate split group 412a and a split group score 414b for the candidate split group 412b. In some cases, the split group score 414b is greater than the split group score 414a. As indicated below, the better split group score can affect an order of determining (and scoring) a next candidate split group.

In some embodiments, the candidate split group 412a and the candidate split group 412b represent partial split groups. Generally, a partial split group comprises one or more fragment alignments that represent fragment alignments for a part but not the whole nucleotide read. The split-read alignment system 106 can link additional fragment alignments to a partial split group. For example, in some embodiments, the split-read alignment system 106 links additional fragment alignments to partial split groups with the highest split group score as part of dynamic programming. By linking additional fragment alignments to highest-scoring partial split groups, the split-read alignment system 106 reduces the processing power required to exhaustively generate candidate split groups.

Although not shown in FIG. 4, after grouping (and determining a split group score for) the fragment alignment 410 and the fragment alignment 406 as the candidate split group 412b, the split-read alignment system 106 groups (and determines a split group score for) an additional candidate split group comprising the fragment alignment 410, the fragment alignment 408, and the fragment alignment 406. If the split group score 414b for the candidate split group 412b exceeds an additional split group score for the additional candidate split group, the split-read alignment system 106 continues to group (and determines split group scores for) candidate split groups comprising the fragment alignment 410 and other combinations of fragment alignments. For instance, the split-read alignment system 106 (i) groups (and determines a split group score for) the fragment alignment 410, the fragment alignment 406, and the fragment alignment 404 and (ii) groups (and determines a split group score for) the fragment alignment 410 and the fragment alignment 404.

Additionally, as part of considering candidate split groups, the split-read alignment system 106 can also consider single fragment alignments. As explained above, in some embodiments, the split-read alignment system 106 also considers single fragment alignments following an order of outermost fragment alignments to innermost fragment alignments. Before or after considering the candidate split group 412a, for instance, the split-read alignment system 106 can identify a candidate partial split group comprising the fragment alignment 410. The split-read alignment system 106 generates a partial split group score for the fragment alignment 410. The split-read alignment system 106 subsequently compares the partial split group score with other split group scores, such as the split group score 414a for the candidate split group 412a. In addition to candidate split groups comprising a new or additional fragment alignment, therefore, in some embodiments, the split-read alignment system 106 also identifies (and determines a split group score for) candidate partial split groups comprising the new or additional fragment alignment.

As further illustrated in FIG. 4, the split-read alignment system 106 generates a candidate split group 412n comprising a fragment alignment 402, a fragment alignment 406, and a fragment alignment 410. The split-read alignment system 106 adds the fragment alignment 402 to the candidate split group 412b because, in this example, the candidate split group 412b has the highest scoring split group score, that is, the split group score 414b. The split-read alignment system 106 scores the candidate split group 412n and assigns a split group score 414n. In this manner, the split-read alignment system 106 iterates from outermost fragment alignments toward innermost fragment alignments. For each fragment considered, the split-read alignment system 106 finds the best next fragment alignment (that is, the next, outer-ward, highest-scoring fragment alignment).

If adding the next outer-ward fragment alignment results in an improved split group score, the split-read alignment system 106 retains the next outer-ward fragment alignment as part of a candidate split group. If adding the next outer-ward fragment alignment does not result in an improved split group score, the split-read alignment system 106 discards the next outer-ward fragment alignment from the candidate split group and moves forward to a yet next outer-ward fragment alignment. By performing dynamic programming, the split-read alignment system 106 accordingly continues to group (and determine split group scores for) candidate split groups following the order of outermost fragment alignments to innermost fragment alignments of a nucleotide read-until each candidate split group is either considered or eliminated as not capable of improving a highest split group score.

As just noted, the split-read alignment system 106 determines split group scores for candidate split groups. FIG. 5 illustrates and the corresponding discussion further detail the split-read alignment system 106 determining split group scores for candidate split groups in accordance with one or more embodiments. In some implementations, the split-read alignment system 106 determines a split group score for a candidate split group based on fragment alignment scores 502, a break penalty 506, and an overlap penalty 508. For example, the split-read alignment system 106 can generate a split group score by combining the fragment alignment scores 502 for fragment alignments within the candidate split group and subtracting the break penalty 506 and the overlap penalty 508 from the combined fragment alignment scores.

As mentioned above, the split-read alignment system 106 can assign each candidate split group a split group score. In some embodiments, a candidate split group comprises any chain of fragment alignments following certain rules. For instance, candidate split groups comprise chains of one or more fragment alignments for the same read from a head fragment to a tail fragment. Under one embodiment of rules, the head fragment is closest to the inner end of the nucleotide read and the tail fragment closest to the outer end of the nucleotide read. A fragment's inner gap is its distance from the nucleotide read's inner end, and a fragment's outer gap is its distance to the nucleotide read's outer end. For consecutive fragment alignments A and B, for example, the rules can be represented as follows: (i) A,inner_gap≤B.inner_gap and (ii) A.outer_gap>B.outer_gap. The same fragment alignment may participate in multiple split groups.

As illustrated in FIG. 5, the split-read alignment system 106 generates the fragment alignment scores 502 for fragment alignments A and B. As indicated above, a fragment alignment score can include a numeric score, metric, or other quantitative measurement of an alignment accuracy of a fragment alignment from a nucleotide read. For instance, a fragment alignment score may indicate the likelihood that a given alignment of a fragment is correct with respect to a reference genome. As indicated above, such a fragment alignment score can indicate a probabilistic degree to which the nucleobases if a nucleotide-read fragment match or are similar to a reference sequence (or an alternate contiguous sequence) from a reference genome. For instance, the split-read alignment system 106 may assign fragment alignment scores to individual fragment alignments within a split group by determining a Smith-Waterman score or a version of a Smith-Waterman score. In other implementations, the split-read alignment system 106 utilizes variations of fragment alignment scoring. As illustrated, the split-read alignment system 106 combines (e.g., sums) fragment alignment scores of the two fragment alignments A and B within the split group.

As further illustrated in FIG. 5, the split-read alignment system 106 determines the break penalty 506. FIG. 5 illustrates three factors that the split-read alignment system 106 analyzes to generate the break penalty 506-a fragment alignment orientation, same reference sequence, and effective indel length. As suggested above, in some embodiments, the break penalty 506 represents a metric that penalizes fragments alignments of a split group to a degree the relative geometries of the fragment alignments exhibit a break of nucleobases. More specifically, the break penalty 506 indicates a relative geometry of the fragment alignments A and B with respect to the reference genome. In some embodiments, and as illustrated in FIG. 5, the split-read alignment system 106 determines the break penalty 506 based on fragment alignment orientation. For example, fragment alignment orientation refers to whether fragment alignments have forward or reverse orientations. To illustrate, in some cases, an expected orientation of a paired-end template would be two fragment alignments pointing toward each other. For instance, the split-read alignment system 106 determines the break penalty 506 based on whether fragment alignments A and B have opposite orientations or are inverted.

In some implementations, the split-read alignment system 106 determines an inversion penalty (e.g., represented as split-inv-pen) if fragment alignments A and B have opposite orientations. If fragment alignments A and B do not have opposite orientations, the split-read alignment system 106 does not assign such an inversion penalty.

Additionally, and as illustrated in FIG. 5, the split-read alignment system 106 determines the break penalty 506 based on whether the fragment alignments are located in the same reference sequence of the reference genome. To illustrate, the split-read alignment system 106 may associate a maximum break penalty (e.g., represented as “split-max-pen”) if fragment alignments A and B are aligned to different reference sequences of the reference genome. The maximum break penalty may comprise predetermined values for DNA and RNA. For example, based on determining that fragment alignments A and B are aligned to different reference sequences, the split-read alignment system 106 assigns a 36-point penalty for DNA and a 20-point penalty for RNA fragment alignments when determining a split-group score. If fragment alignments A and B are aligned to the same reference sequence, in some embodiments, the split-read alignment system 106 calculates an effective indel length (indelLen) as the absolute difference between the fragment alignments' alignment diagonals at their facing ends.

As further illustrated in FIG. 5, the split-read alignment system 106 determines the break penalty 506 based on effective indel length. In some implementations, the split-read alignment system 106 reduces the break penalty 506 based on the indel length. For instance, the split-read alignment system 106 can reduce the overlap penalty by MIN(overlap, FLOOR(Log 4(indelLen)), split-olap-ignore). In some implementations, indelLen equals an indel length measured in nucleobase pairs. The split-read alignment system 106 reduces the overlap penalty because (a) overlap implies similar sequences at fragment alignments A and B, which is common for SV breaks, and (b) much of the penalty for long-distance breaks comes from the exponentially large number of potential breakend positions. But the number of potential breakend positions is reduced to a smaller set when considering only breakend positions with sufficient sequence similarity to cause the fragment overlap.

In some implementations, the split-read alignment system 106 may limit or disable overlap reduction by setting a split-olap-ignore value lower or to zero. When allowing overlap reduction, the split-read alignment system 106 may set split-log 2-coeff of at least 0.5 so that overlapping breaks do not receive penalties reducing, rather than increasing, with distance.

Instead of determining the effective indel length, in some embodiments, the split-read alignment system 106 determines a break distance in a chromosome. In one example, the split-read alignment system 106 determines the distance between fragment alignment start points within the reference genome and compares the distance between fragment alignment start points with an expected break distance. In another example, the split-read alignment system 106 determines a distance between the nearest endpoints of two fragment alignments and compares the distance with an expected break distance.

Furthermore, in split alignment instances, the split-read alignment system 106 determines an initial break penalty (e.g., represented as split-open-pen) before considering an effective indel length. In at least one example, the break penalty equals the greater of (i) the maximum break penalty or (ii) a break penalty determined based on an inversion penalty (invPen) and an indel Length (indelLen). To illustrate, the break penalty equals MIN(split-max-pen, split-open-pen+invPen+FLOOR(split-log 2-coeff*Log 2(indelLen))).

FIG. 5 further illustrates the split-read alignment system 106 determining an overlap penalty 508. As suggested above, in some embodiments, the overlap penalty 508 represents a metric that penalizes fragment alignments of a split group at a degree to which the fragment alignments overlap within the nucleotide read. For instance, in some embodiments, the overlap penalty 508 equals the amount of overlap within the read between fragment alignments A and B times a Smith-Waterman match-score. As indicated above, fragment-alignment overlap can occur when fragments include (and align with a reference genome) overlapping nucleotide-read bases from a nucleotide read. By determining an overlap penalty, the split-read alignment system 106 avoids double-counting read nucleobases that match the reference genome within both fragments of fragment alignments.

In some implementations, the split-read alignment system 106 further determines other penalties as part of determining a split group score. To illustrate, the split-read alignment system 106 may determine a gap penalty. A gap penalty is complementary to the overlap penalty 508. More particularly, in some embodiments, a gap penalty represents a numeric score, metric, or other quantitative measurement that penalizes fragment alignments of a split group to a degree to which a gap exists between the fragment alignments. In some implementations, the gap penalty represents a negative overlap, and the overlap penalty represents a negative gap.

As mentioned above, in some embodiments, the split-read alignment system 106 generates and scores split groups by using dynamic programming. Accordingly, in some embodiments, the split-read alignment system 106 generates split group scores for candidate split groups as illustrated in FIG. 5 following an order of outermost fragment alignments toward innermost fragment alignments as illustrated in FIG. 4.

In some implementations, and as previously mentioned, the split-read alignment system 106 evaluates candidate split groups based on pair scores. More specifically, the split-read alignment system 106 evaluates pair alignments of candidate pairs of split groups and selects a predicted split group based on pair scores. FIG. 6A illustrates the split-read alignment system 106 generating pair scores in accordance with one or more embodiments. FIG. 6B illustrates the split-read alignment system 106 determining a predicted split group based on the pair scores in accordance with one or more embodiments.

FIG. 6A illustrates the split-read alignment system 106 generating a pair score based on split group scores 602 and a pairing penalty 608. In some implementations, the split-read alignment system 106 identifies, from the candidate split groups, candidate pairs of split groups comprising different fragment alignments for mates of a paired-end nucleotide read. For example, the split-read alignment system 106 identifies a candidate pair of split groups comprising a split group 604 and a split group 606. More specifically, the split group 604 comprises fragment alignments A and B, and the split group 606 comprises fragment alignments C and D. As shown, the split group 604 and the split group 606 are aligned with the reference genome. More specifically, the split group 604 and the split group 606 comprise candidate paired-end mates aligned along a reference genome. For instance, the split group 604 may represent R1 and the split group 606 can represent R2 of a paired-end read.

As further illustrated in FIG. 6A, the split-read alignment system 106 generates the split group scores 602. As suggested above, in some embodiments, the pair scores evaluate an accuracy of pair alignments of the candidate pairs of split groups with the reference genome. In some implementations, the split group scores 602 comprise the sum of the split group scores of the candidate pairs of split groups. To illustrate, the split-read alignment system 106 sums a split group score for the split group 604 and a split group score for the split group 606 as some or all of a pair score.

As further illustrated in FIG. 6A, the split-read alignment system 106 generates the pairing penalty 608 for the candidate pair of split groups. The split-read alignment system 106 may determine the pairing penalty 608 based on an estimated insert size between innermost fragment alignments of the candidate pairs of split groups. In some cases, fragment alignments corresponding to paired-end mates are located relatively close to each other in the reference genome. The split-read alignment system 106 can determine a known empirical insert size distribution. In some embodiments, the split-read alignment system 106 determines the known empirical insert size distribution by analyzing insert sizes in a sequence library. The known empirical insert size distribution generally indicates most likely insert sizes of a sequence library. Accordingly, the split-read alignment system 106 may assign a zero or small pairing penalty if the two innermost fragment alignments are located close together or an expected distance from each other based on the empirical insert size distribution.

For example, the split-read alignment system 106 determines an estimated insert size 610 between innermost fragment alignments B and C. As indicated by FIG. 6A, the estimated insert size 610 comprises a length of the library template from which the mate nucleotide reads were sequenced at each end. The split-read alignment system 106 compares the estimated insert size 610 with an expected insert size based on the empirical insert size distribution. The split-read alignment system 106 assigns a greater pairing penalty for candidate pairs of split group where the estimated insert size 610 is greater or even smaller than an expected insert size. In some embodiments, the split-read alignment system 106 determines a fixed pairing penalty for candidate pairs of split groups outside an expected insert size range. In other implementations, the split-read alignment system 106 utilizes a sliding scale where the split-read alignment system 106 modulates the pairing penalty based on a difference between the estimated insert size 610 and an expected insert size.

In some examples, the estimated insert size is calculated to reflect the estimated total length of the library template strand that was sequenced at each end to obtain two paired-end nucleotide reads. For instance, the two paired-end nucleotide reads comprise the fragment alignments A, B, C, and D. In at least one implementation, the insert size is estimated from the reference positions of the endpoints of the innermost fragment alignments B and C and extrapolated to account for outer portions of the two paired-end nucleotide reads not covered by the fragment alignments B and C. To illustrate, the split-read alignment system 106 can extrapolate to account for outer portions including portions covered by the fragment alignments A and D. However, in the example illustrated in FIG. 6A, the split-read alignment system 106 does not consider the reference positions of outer fragment alignments A and D because of the SV break between the fragment alignments A and B and the SV break between the fragment alignments C and D. Because of the SV breaks, accordingly, the locations of the fragment alignments A and D are not strongly informative about the true insert size.

In some implementations, the split-read alignment system 106 further adjusts the pairing penalty 608 based on split group locations and split group orientations. For example, the split-read alignment system 106 can assign a greater pairing penalty for split groups in a candidate pair of split groups that are aligned to different chromosomes of a reference genome. As mentioned, the split-read alignment system 106 may also assign greater pairing penalties based on the orientations of the split groups. For instance, if fragment alignments are oriented in the same orientation (e.g., both oriented from 3′ to 5′ of a reference genome) rather than complimentary orientations (e.g., pointing toward each other), the split-read alignment system 106 assigns a greater pairing penalty to the candidate pair of split group.

In one or more embodiments, the split-read alignment system 106 determines pair scores based on the split group scores 602 and the pairing penalty 608. To illustrate, in some implementations, the split-read alignment system 106 generates a pair score by subtracting the pairing penalty 608 from a sum of the split group scores 602.

As mentioned, in some cases, two paired-end mate reads overlap the same breakpoint (e.g., SV breakpoint). When overlapping mates cross a breakpoint in their overlap zone, each mate may be split aligned similarly, as two fragment alignments each. In some embodiments, the split-read alignment system 106 detects these “quads” as a special case and assigns pair scores involving only one copy of the break penalty (but both overlap penalties). When such a “quad” of split overlapping alignments exhibits a highest pair score, the split-read alignment system 106 selects R1 and R2 fragment alignments on the same side of the break as primary alignments, that is, one 5′ fragment alignment and one 3′ fragment alignment, to support proper pairing. Generally, the split-read alignment system 106 selects the higher-scoring 5′ fragment alignment as a primary alignment along with the mate's 3′ fragment alignment.

In some embodiments, detection of quads is somewhat restrictive. Corresponding fragments in both mates need to be clipped at the SV break at identical positions, which typically occurs unless sequencing errors intervene. Gaps or overlap between fragments in each nucleotide read is allowed but they must be the same in both mates of a paired-end read. If the split-read alignment system 106 cannot detect a perfect quad, the split-read alignment system 106 outputs only three fragment alignments, omitting the lowest-scoring 3′ fragment alignment.

As mentioned, in some embodiments, the split-read alignment system 106 selects predicted split groups based on pair scores. FIG. 6B illustrates and the corresponding paragraphs describe the split-read alignment system 106 selecting predicted split groups based on pair scores. By way of overview, FIG. 6B illustrates pair scores 622 for candidate pairs of split groups 626a-626c. The candidate pair of split groups 626a comprises a split group 611 and a split group 612. The empty box within the fragmented arrow in the split group 612 represents a break (e.g., an SV break) between the fragment alignments that make up the split group 612. In contrast to the candidate pair of split groups 626a, the candidate pair of split groups 626b comprises a split group 614 and a split group 616. Finally, the candidate pair of split groups 626c comprises a split group 618 and a split group 620. As explained below, the split-read alignment system (i) selects a pair of candidate split groups having a highest pair score and (ii) selects a predicted split group for each mate of a nucleotide-read pair from the pair of candidate split groups having the highest pair score.

In some cases, the candidate split group with the highest split group score may not necessarily exhibit a correct split alignment. For instance, a relatively higher split group score indicates a likely way that a nucleotide read exhibits a split alignment. However, this relatively higher split group score may involve an unlikely pairing configuration of two mates from a pair of paired-end nucleotide reads. By generating a pair score in addition to a split group score, the split-read alignment system 106 further considers pairing configurations of fragment alignments from mates of paired-end nucleotide reads when selecting a predicted split group.

To illustrate, for instance, the split group 614 may have the highest split group score of the split groups 611-620. The split-read alignment system 106 generates the pair scores 622 for the candidate pairs of split groups 626a-626c. Based on determining that the pair score for the candidate pair of split groups 626a exceeds the pair score for the candidate pair of split groups 626b, in some cases, the split-read alignment system 106 selects the split group 611 from the candidate pair of split groups 626a as the predicted split group for a particular mate instead of the split group 614 from the candidate pair of split groups 626b.

In some implementations, the split-read alignment system 106 generates a fragment alignment mapping score (e.g., MAPQ) corresponding to a fragment alignment corresponding with the highest pair score. The fragment alignment mapping scores represent a confidence that a given fragment alignment is part of a true alignment from the perspective of a mapping-quality metric (e.g., MAPQ). The fragment alignment mapping score for one fragment alignment is not conditional on other fragment alignments. The fragment alignment mapping score is rather proportional to the difference between the highest pair score and the next-highest pair score that did not involve the fragment alignment of interest.

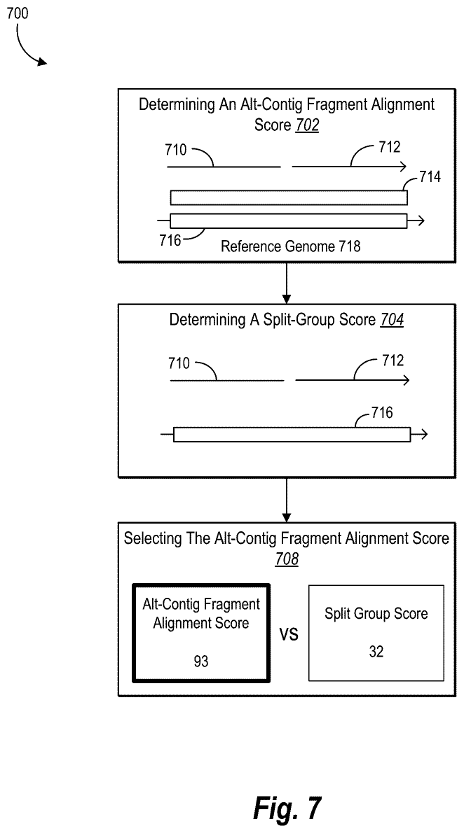

In some implementations, the split-read alignment system 106 may determine fragment alignments align with alternate contiguous (or “alt-contig”) sequences within the reference genome. FIG. 7 illustrates the split-read alignment system 106 scoring alt-contig fragment alignments that correspond to nucleotide reads with alternate contiguous sequences in accordance with one or more embodiments. By way of overview, FIG. 7 illustrates a series of acts 700 comprising an act 702 of determining an alt-contig fragment alignment score, an act 704 of determining a split-group score, and an act 708 of selecting the alt-contig fragment alignment score. If the alt-contig fragment alignment score exceeds the split group score for the fragment alignments, the split-read alignment system 106 reports a split alignment of fragment alignments with the primary assembly that correspond to the alternate contiguous sequence.

Generally, the split-read alignment system 106 identifies an alternate contiguous sequence representing a structural variant. The split-read alignment system 106 determines that fragments of a nucleotide read exhibit highest fragment alignment scores with the alternate contiguous sequence and accordingly reports a split alignment in the corresponding primary-assembly region. If, for instance, the split-read alignment system 106 determines that a split alignment for a nucleotide read exhibits an alt-contig fragment alignment score with respect to an alternate contiguous sequence—where the alt-contig fragment alignment score exceeds split group scores for other candidate split groups for the nucleotide reads—the split-read alignment system 106 uses the alt-contig fragment alignment score for the liftover-corresponding split alignment (without any break penalty) instead of the other candidate split group scores. Thus, the alt-contig fragment alignment score may guide the split-read alignment system 106 to select and report a given split alignment over other candidate split alignments represent by other split groups that may have otherwise scored better in the absence of the alt-contig fragment alignment score.

When an alternate contiguous sequence represents an SV breakpoint, for example, the split-read alignment system 106 can recognize two primary fragment alignments for a same liftover group as one alternate fragment alignment. In some cases, multiple primary fragments for one liftover group are treated as duplicates of each other and only the best scoring fragment alignment is retained. However, in the case of a nucleotide read matching an alternate contiguous sequence spanning an SV break, the split-read alignment system 106 can retain both primary fragment alignments and join them into a split group that uses an alternate contiguous sequence's alignment score.

As shown in FIG. 7, the series of acts 700 illustrate the split-read alignment system 106 using a scoring system to identify a split alignment that represents a structural variant when detecting an alt-contig fragment alignment. Generally, the split-read alignment system 106 determines when a liftover group has two primary fragment alignments—a 5′ fragment alignment and a 3′ fragment alignment—that extend beyond each other in a nucleotide read. A liftover group comprises fragment alignments that align with either a primary-assembly region or alternate contiguous sequence for the same genomic region of reference genome. In some implementations, the split-read alignment system 106 determines that the 5′ fragment alignment and the 3′ fragment alignment exhibit alt-contig properties.

To identify such alt-contig fragment alignments, in some embodiments, the split-read alignment system 106 determines a split-alternate-minimum extension (split-alt-min-ext) by which two primary fragment alignments must extend beyond each other in a nucleotide read. The split-read alignment system 106 uses a split-alt-min-ext to identify fragment alignments qualifying as alt-contig fragment alignments. In some implementations, the split-alt-min-ext comprises a predetermined value (e.g., 20 bases); in other implementations, the split-read alignment system 106 determines split-alt-min-ext based on user input. In general, a higher split-alt-min-ext is more restrictive, making it less likely that the split-read alignment system 106 identifies alt-contig fragment alignments. In some embodiments, the split-read alignment system 106 sets the split-alt-min-ext to 0 to disable liftover-guided split alignment. For example, the 5′ fragment alignment must begin within the first split-alt-min-ext bases of the nucleotide read. 5′ fragment must extend at least split-alt-min-ext bases toward the 5′ end than the 3′ fragment. The 3′ fragment must extend at least split-alt-min-ext bases toward the 3′ end than the 5′ fragment. The best-scoring alignment in the liftover group must be an alt-contig alignment.

To determine whether fragment alignments with an alternate contiguous sequence score better than other candidate split groups for a nucleotide read, the split-read alignment system 106 can use the scoring approach depicted in FIG. 7. As illustrated in FIG. 7, the split-read alignment system 106 performs the act 702 of determining an alt-contig fragment alignment score. The split-read alignment system 106 determines an alt-contig fragment alignment score for an inner fragment alignment 712 (the 3′ fragment) and an outer fragment alignment 710 (the 5′ fragment) corresponding to a nucleotide read. As indicated by FIG. 7, both the inner fragment alignment 712 and the outer fragment alignment align with an alternate contiguous sequence 714 within a reference genome 718. The alternate contiguous sequence 714 comprises an alternate sequence for a primary-assembly region 716 of the reference genome. Contrary to some existing sequencing systems, the split-read alignment system 106 does not consider the inner fragment alignment 712 and the outer fragment alignment 710 to be duplicates, but allows both to participate separately for purposes of scoring-provided that they satisfy requirements that the minimum length that two fragment alignments must extend beyond each other in the read (e.g., requirements of split-alt-min-ext).

Indeed, in some embodiments, the split-read alignment system 106 determines an alt-contig fragment alignment score for the inner fragment alignment 712 and an alt-contig fragment alignment score for the outer fragment alignment 710 in the same way the split-read alignment system 106 determines fragment alignment scores. For instance, the split-read alignment system 106 determines the alt-contig fragment alignment scores by determining a Smith-Waterman score or variations of a Smith-Waterman score.

In addition to determining an alt-contig fragment alignment score for each of the fragment alignments, the split-read alignment system 106 performs the act 704 of determining a split-group score. In particular, the split-read alignment system 106 determines a split group score for the inner fragment alignment 712 and the outer fragment alignment 710 with a primary-assembly region 716 of the reference genome 718.

As further shown in FIG. 7, the split-read alignment system 106 further performs the act 708 of selecting the alt-contig fragment alignment score. Generally, the split-read alignment system 106 utilizes the liftover group's best alignment score among the alt-contig fragment alignment score(s) and the split group score. Accordingly, the split-read alignment system 106 may replace a split group score with the best alt-contig fragment score. Accordingly, the alt-contig fragment score becomes a replacement split group score.

Based on determining that the alt-contig fragment alignment score exceeds the split group score, the split-read alignment system 106 utilizes the alt-contig fragment alignment score in fragment alignment processing. In some embodiments, the split-read alignment system 106 further compares and determines that the alt-contig fragment alignment score exceeds other split group scores of the inner fragment alignment and the outer fragment alignment with other primary-assembly regions.

If the alt-contig fragment alignment score exceeds the split group score for the fragment alignments, the split-read alignment system 106 reports the associated split alignment comprising the outer fragment alignment 710 and the inner fragment alignment 712. By reporting the associated split alignment, the split-read alignment system 106 effectively reports or indicates an alignment of the nucleotide read with the alternate contiguous sequence 714 itself. By utilizing the alt-contig fragment alignment score as a replacement split group score, the split-read alignment system 106 facilitates the selection of the split group corresponding to the alternate contiguous sequence 714 over other candidate split groups. In other words, the split-read alignment system 106 grants a split group for a primary assembly a higher score inherited from an alt-contig sequence corresponding with the primary assembly. By using the alt-contig fragment alignment score as a split group score, the split-read alignment system 106 further increases the fragment alignment mapping score (e.g., MAPQ) corresponding to fragment alignments within the split group.

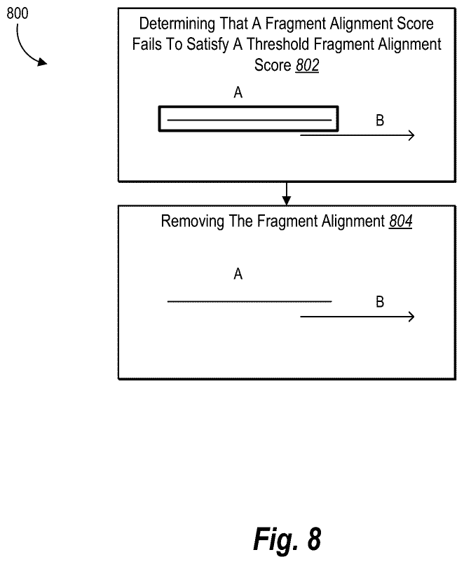

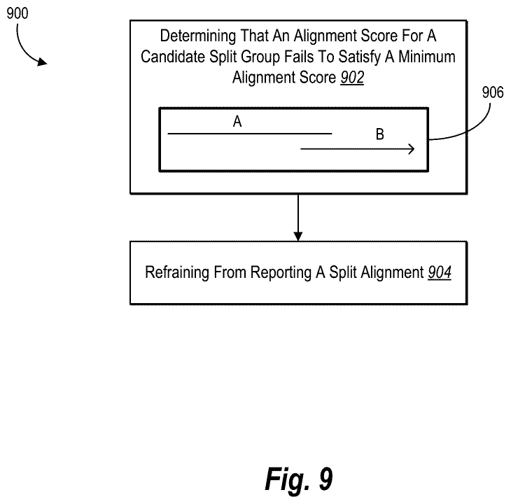

In some embodiments, the split-read alignment system 106 filters unreliable fragment alignments by utilizing a threshold fragment alignment score and a minimum alignment score. In accordance with one or more embodiments, FIGS. 8-9 illustrate the split-read alignment system 106 utilizing a threshold fragment alignment score and a minimum alignment score to remove candidate split groups and identify candidate split groups on which to not report alignments, respectively.

FIG. 8 illustrates the split-read alignment system 106 utilizing a fragment alignment score remove malformed candidate split groups in accordance with one or more embodiments. By way of overview, FIG. 8 illustrates a series of acts 800 including an act 802 of determining that a fragment alignment score fails to satisfy a threshold fragment alignment score and an act 804 of removing the fragment alignment.

As illustrated in FIG. 8, the series of acts 800 includes the act 802 of determining that a fragment alignment score fails to satisfy a threshold fragment alignment score. In particular, the split-read alignment system 106 determines that a fragment alignment score for a fragment alignment corresponding to a candidate split group fails to satisfy a threshold fragment alignment score. The split-read alignment system 106 may determine the threshold fragment alignment score based on user input. Additionally, or alternatively, the split-read alignment system 106 generates a predetermined fragment alignment score. The threshold fragment alignment score may comprise a minimum fragment alignment score for a fragment alignment to participate in a split-read alignment. For example, the fragment alignment score for the fragment alignment A may fall below the threshold fragment alignment score.

As further illustrated in FIG. 8, the split-read alignment system 106 performs the act 804 of removing the fragment alignment. More particularly, the split-read alignment system 106 removes sub-threshold fragment alignments from consideration in forming candidate split groups. For instance, based on determining that the fragment alignment score for the fragment alignment A falls below the threshold fragment alignment score, the split-read alignment system 106 removes the fragment alignment A from consideration. Accordingly, the split-read alignment system 106 never forms a split group comprising the fragment alignment A and the fragment alignment B. By removing sub-threshold fragment alignments from consideration, the split-read alignment system 106 effectively filters unreliable fragment alignments at the input, ignoring them completely. A threshold fragment alignment score is mainly useful for low-scoring inner (3′) fragments which could be included because their properly paired positions capture large score benefits via low pairing penalties. Furthermore, in some implementations, the split-read alignment system 106 also blocks sub-threshold fragment alignments from participating in any generated multi-fragment-alignment split groups.

The split-read alignment system 106 further reduces noise by utilizing a minimum alignment score. FIG. 9 illustrates the split-read alignment system 106 utilizing a minimum alignment score to identify candidate split groups on which to not report alignments in accordance with one or more embodiments. By way of overview, FIG. 9 illustrates a series of acts 900 including an act 902 of determining that an alignment score for a candidate split group fails to satisfy a minimum alignment score and an act 904 of refraining from reporting a split alignment.

As illustrated in FIG. 9, the split-read alignment system 106 performs the act 902 of determining that an alignment score for a candidate split group fails to satisfy a minimum alignment score. The alignment score for the candidate split group refers to an alignment score for an entire split group. In some implementations, the alignment score for the candidate split group comprises a split group score. As an example, the split-read alignment system 106 determines that the split group score for a candidate split group 906 falls below a minimum alignment score. The split-read alignment system 106 may determine the minimum alignment score based on user input or may predetermine the minimum alignment score.

In contrast with existing sequencing systems, the split-read alignment system 106 may report split alignments, even if the component fragment alignments have low fragment alignment scores. To illustrate, a fragment alignment A and/or a fragment alignment B may have individual alignment scores below the minimum alignment score; however, the A+B split group score may be higher, and exceed, the minimum alignment score. In this case, the split-read alignment system 106 may report the A+B split alignment. By contrast, existing sequencing systems would have filtered out one or both of the fragment alignment A and/or the fragment alignment B for not meeting the minimum alignment score. Essentially, the split-read alignment system 106 leverages the generation of split group scores by splitting a threshold score into two separate parameters—the threshold fragment alignment score and the minimum alignment score. The threshold fragment alignment score filters fragment alignments up front by disqualifying sub-threshold fragment alignments from participating in split alignments. The threshold fragment alignment score utilized by the split-read alignment system 106 may be higher and more forgiving than alignment scores utilized by existing sequencing systems. In some embodiments, the split-read alignment system 106 configures the minimum alignment score to filter candidate split groups only after low-scoring fragment alignments have had opportunities to participate in candidate split groups that may potentially achieve higher split group scores. Thus, the split-read alignment system 106 retains a final minimum score achieving a similar target level of noise filtering as existing sequencing systems but in a way that provides sensitivity to lower-scoring constituent fragment alignments being part of full-read alignments.

The split-read alignment system 106 additionally performs the act 904 of refraining from reporting a split alignment. In particular, the split-read alignment system 106 refrains from reporting a split alignment of the candidate split group in an alignment file or a variant call file based on the alignment score failing to satisfy the minimum alignment score. To illustrate, the split-read alignment system 106 does not report the candidate split group 906 as a predicted split group.

In some embodiments, even though the split-read alignment system 106 does not report the candidate split group 906, the split-read alignment system 106 still considers the candidate split group 906 as competition for other alignments. If the highest pair score involves a split-group score below the minimum alignment score, then the split-read alignment system 106 returns the read unmapped. But even if another alignment or split group exhibits the highest pair score, the split-read alignment system 106 may reduce a fragment alignment mapping score (e.g., MAPQ) for the fragment alignment if the pair score of the failing split group was second best. As mentioned, the fragment alignment mapping score represents a confidence that a given fragment alignment is part of (or mapped to) a true alignment from the perspective of a mapping-quality metric (e.g., MAPQ).

In some implementations, the split-read alignment system 106 generates and stores configuration registers as part of determining split-read alignments. The previous discussion described register entries, including split-log 2-coeff, primary-5p, and others. The following table provides an overview of additional configuration register entries defined by the split-read alignment system 106 in accordance with one or more embodiments.

| Name | | | |
|---|---|---|---|
| (--Aligner.XXX | DNA Default | RNA Default | Description |
| Primary-5p | 0 | 0 | Set to emit 5′-most |
| | | | fragment alignments of |
| | | | split alignments as |
| | | | primary, rather than |
| | | | properly-paired |
| | | | fragment alignments |
| | | | (normally 3′) |
| Split-secondary | 0 | 0 | Set to enable split |
| | | | secondary alignments, |
| | | | yielding records with |
| | | | both secondary and |
| | | | supplementary flags |
| Split-local-dist | 0xFFFFFFFF | 0xFFFFFFFF | For split alignment |
| | | | break penalties, |
| | | | maximum effective |
| | | | indel length considers |
| | | | ‘local,’ receiving a |
| | | | sub-maximum penalty |
| Split-inv-pen | 4 | 4 | For split alignment, |
| | | | extra break penalty for |
| | | | change in orientation |
| | | | (inversion) |
| Split-open-pen | 8 | 4 | For split alignment, |
| | | | initial break penalty |
| | | | before considering |
| | | | effective indel length |
| Split-log-2-coeff | 0.875 | 0.5 | For split alignment |
| | | | break penalties, value |
| | | | multiplied by log2 of |
| | | | the effective indel |
| | | | length |
| Split-max-pen | 36 | 20 | Maximum split |
| | | | alignment break |
| | | | penalty |
| Frag-min-score | 12 | 12 | Minimum score for |
| | | | fragment alignment to |
| | | | participate in split-read |
| | | | alignment. This can be |
| | | | lower than aln-min- |
| | | | score, which applies to |
| | | | complete split-read |
| | | | scores |
| Split-alt-min-ext | 20 | 20 | For alt-liftover guided |
| | | | split alignment, |
| | | | minimum length two |
| | | | primary fragment |
| | | | alignments must |
| | | | extend beyond each |
| | | | other in the read |
| Split-olap-ignore | 16 | 16 | Maximum fragment |
| | | | alignment overlap |
| | | | unpenalized, for inter- |
| | | | chromosome breaks, |
| | | | or up to log4 of the |
| | | | effective indel length |
| | | | intra-chromosome |

In some implementations, the split-read alignment system 106 assigns alignment tags to fragment alignments denoting strand orientation. More specifically, an XS tag is defined as a raw competing fragment score. In some implementations, XS for a given fragment alignment is the highest score of any other fragment alignment mostly overlapping the given fragment alignment from the nucleotide read (and hence is not eligible for split alignment with the given fragment alignment). In other embodiments, the split-read alignment system 106 determines the XS for all non-secondary fragment alignments (both primary and supplementary) is the highest fragment score not involved in the winning or highest scoring split group. XS for all secondary alignments (both non-supplementary and supplementary) is the highest fragment score involved in the winning split group.

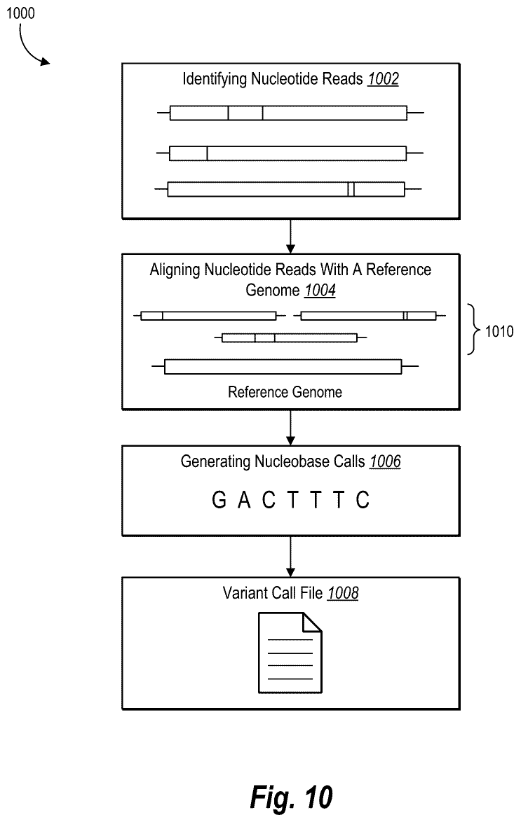

In some embodiments, the split-read alignment system 106 determines nucleobase calls for a genomic region based on an alignment of the predicted split group with a reference genome. FIG. 10 illustrates the split-read alignment system 106 generating nucleobase calls and a variant call file in accordance with one or more embodiments. By way of overview, FIG. 10 illustrates a series of acts 1000 including an act 1002 of identifying nucleotide reads, an act 1004 of aligning nucleotide reads with a reference genome, an act 1006 of generating nucleobase calls, and a resulting variant call file 1008.

As illustrated in FIG. 10, the split-read alignment system 106 performs the act 1002 of identifying nucleotide reads. In one or more embodiments, the act 1002 comprises identifying nucleotide reads from a genomic sample. In some implementations, the sequencing device 114 determines nucleotide reads from the sample genome (e.g., by using SBS) and sends the data representing the nucleotide reads (e.g., in a base-call file) to the sequencing system 104. In alternative implementations, a third-party system determines the nucleotide reads from the sample genome and allows the sequencing system 104 access to the nucleotide reads.

The series of acts 1000 illustrated in FIG. 10 further includes the act 1004 of aligning nucleotide reads with a reference genome. As illustrated, the split-read alignment system 106 aligns the nucleotide reads 1010 with a reference genome. For example, in various implementations, the sequencing system 104 aligns the nucleotide reads 1010 with the reference genome. As part of performing the act 1004, the split-read alignment system 106 determines fragment alignments and determines predicted split groups.

As further illustrated in FIG. 10, the split-read alignment system 106 performs the act 1006 of generating nucleobase calls. Generally, the nucleobase calls include a prediction of a nucleobase at a genomic coordinate of the sample genome for the variant call file 1008 (VCF) or other base-call-output file based on aligning nucleotide reads to the reference genome. Because of the accuracy of predicted split groups, the sequencing system 104 can generate the nucleobase calls with more accuracy and confidence for genomic coordinates than existing sequencing systems.

In some examples, the split-read alignment system 106 reports split alignments using BAM/SAM file formats. The BAM/SAM file specification provides for three different alignment types: primary, supplementary, and secondary. In some examples, FLAG bits indicate supplementary and/or secondary designations. According to BAM/SAM specifications, exactly one primary alignment is recognized (having neither supplementary or secondary FLAG sets). A split alignment with N>=2 fragments is accordingly represented as 1 primary fragment alignment BAM/SAM record, and N−1 supplementary fragment alignment BAM/SAM records.

Thus, ordinarily, the split-read alignment system 106 may not output the whole split group as a primary alignment unless by special means or encoding. The split-read alignment system 106 identifies which of the N fragment alignments should be selected for primary alignment status, the remaining N−1 fragment alignment receiving supplementary alignment status. In some implementations, the split-read alignment system 106 determines the primary alignment output based on parameter primary-5p. When primary-5p=0, primary fragment alignments are selected to support proper paring, normally, the 3′-most fragment alignments. Additionally, or alternatively, the split-read alignment system 106 sets primary-5p to 1 to set the 5′-most fragment alignment as the primary alignment.

If the split-read alignment system 106 determines to output secondary alignments, the split-read alignment system 106 selects secondary fragment alignments in decreasing order of pair score. Generally, secondary alignments comprise an additional alignment record that is not related to the primary alignment but rather represents an alternative alignment candidate. Some of the secondary fragment alignments may themselves be nontrivial split groups. The split-read alignment system 106 can determine to output full split groups for secondary alignments. Each of the full split groups would mimic the primary/supplementary structure of winning split groups but with secondary flags. However, in instances where fragment alignments of the secondary split-group have already been output (either in the highest-scoring split group or in a higher-scoring secondary split group), the split-read alignment system 106 blocks the output of supplementary secondary fragment alignments. More specifically, supplementary alignments comprise additional alignment records that supplement the primary alignment or present additional parts of a split alignment.

As indicated above, the split-read alignment system 106 improves the alignment of split reads and improves the accuracy of corresponding nucleobase calls, including structural variant calls. In accordance with one or more embodiments, FIGS. 11A-11D illustrate read-pileups for candidate gene fusion events generated by the split-read alignment system 106 that exhibit more accurate mapping and alignment—and result in more accurate variant calling—than an existing sequencing system based on transcriptomic reads. As indicated by FIGS. 11A-11D, by determining split group scores for candidate split groups comprising fragment alignments from fragments of nucleotide reads (e.g., transcriptomic reads) and selecting a predicted split group from among the candidates based on such split group scores, the split-read alignment system 106 (i) identifies fragment alignments for candidate split reads with better accuracy than existing sequencing systems and (ii) determines true-negative variant calls (here, no gene fusion) at genomic coordinates and breakpoints at which existing sequencing systems determine false-positive variant calls for gene-fusion events.

FIGS. 11A and 11B complement one another by depicting a breakpoint along a chromosome (FIG. 11A) and different read fragment alignments and mappings determined by the split-read alignment system 106 and an existing sequencing system (FIG. 11B) with respect to the same breakpoint. As shown in FIG. 11A, for example, a chromosome segment 1102a for chromosome 11 comprises a breakpoint 1104a. In particular, the breakpoint 1104a shown in FIG. 11A identifies one or more genomic coordinates at which nucleotide reads have been aligned by an existing sequencing system with a break between nucleotide-read fragments subsequently depicted in FIG. 11B. As further described below, a split alignment of transcriptomic reads with respect to the breakpoint 1104a can indicate a gene-fusion event for the ARL2-SNX15 RNA gene with another gene.

As shown in FIG. 11B, the user client device 108 presents a graphical user interface 1100a comprising different read fragment alignments and mappings determined by the split-read alignment system 106 and by an existing sequencing system with respect to the breakpoint 1104a. For instance, the graphical user interface 1100a can represent a graphical user interface of Integrative Genomics Viewer (IGV) comprising read alignments with respect to a reference genome. For purposes of comparison, the graphical user interface 1100a comprises an updated alignment window 1106a depicting candidate transcriptomic-read alignments of the split-read alignment system 106, a previous alignment window 1108a depicting candidate transcriptomic-read alignments of an existing sequencing system, and a reference-genome window 1110a depicting reference nucleotide bases of a reference genome. In FIG. 11B, the updated alignment window 1106a also comprises a read coverage marker 1120a that indicates read coverage (e.g., read depth) at genomic coordinates overlapping with the breakpoint 1104a.

As shown in the previous alignment window 1108a, the existing sequencing system maps and aligns transcriptomic read fragments 1114a with the reference genome at genomic coordinates corresponding (or relatively closer) to the breakpoint 1104a. As indicated by the light grey shading of the transcriptomic read fragments 1114a in the previous alignment window 1108a, the called nucleotide bases of the transcriptomic read fragments 1114a match the reference nucleotide bases of the reference genome within the reference-genome window 1110a. In contrast to the transcriptomic read fragments 1114a, the existing sequencing system maps and aligns (i) mismatched transcriptomic read fragments 1112a with a genomic region corresponding to an ARL2 contiguous sequence located upstream from the breakpoint 1104a and (ii) mismatched transcriptomic read fragments 1112b with a genomic region corresponding to an SNX15 contiguous sequence located downstream from the breakpoint 1104a. As indicated by the different grey shading or colors of the mismatched transcriptomic read fragments 1112a and 1112b in the previous alignment window 1108a, the called nucleotide bases of the mismatched transcriptomic read fragments 1112a and 1112b do not match the reference nucleotide bases of the reference genome within the reference-genome window 1110a.

Because a threshold number of called nucleotide bases do not match the reference nucleotide bases, the existing sequencing system clips (e.g., soft clips or hard clips) the nucleotide bases within the mismatched transcriptomic read fragments 1112a and 1112b, thereby ignoring the nucleotide bases of the mismatched transcriptomic read fragments 1112a and 1112b for purposes of alignment. But the mismatched transcriptomic read fragments 1112a and 1112b exhibit split alignments of corresponding transcriptomic reads within respect to the reference genome. Both the candidate alignments of the mismatched transcriptomic read fragments 1112a and 1112b by the existing sequencing system represent supplemental alignments with positive mapping-quality metrics (e.g., positive MAPQ) and correspond to primary alignments with another gene (e.g., AKT3 gene). Based on scoring of the primary and supplemental alignments of such corresponding transcriptomic reads depicted in the previous alignment window 1108a, the existing sequencing system determines a false-positive variant call of a gene-fusion event for a genomic sample. For instance, in some cases, the existing sequencing system re-aligns the mismatched transcriptomic read fragments 1112a and 1112b with genomic regions of another gene on a different chromosome (e.g., AKT3 gene on chromosome 1), thereby indicating a gene-fusion event.

As shown in the updated alignment window 1106a, the split-read alignment system 106 maps and aligns transcriptomic read fragments 1116a with the reference genome at genomic coordinates corresponding (or relatively closer) to the breakpoint 1104a. As indicated by the light grey shading of the transcriptomic read fragments 1116a, the called nucleotide bases of the transcriptomic read fragments 1116a match the reference nucleotide bases of the reference genome within the reference-genome window 1110a. In contrast to the transcriptomic read fragments 1116a, the split-read alignment system 106 maps and aligns mismatched transcriptomic read fragments 1118a with a genomic region corresponding to an SNX15 contiguous sequence located downstream from the breakpoint 1104a, but does not map or align any mismatched transcriptomic read fragments upstream from the breakpoint 1104a. As indicated by the different grey shading or colors of the mismatched transcriptomic read fragments 1118a in the updated alignment window 1106a, the called nucleotide bases of the mismatched transcriptomic read fragments 1118a do not match the reference nucleotide bases of the reference genome within the reference-genome window 1110a.

As further indicated by FIG. 11B, the candidate alignments of the mismatched transcriptomic read fragments 1118a by the split-read alignment system 106 exhibit mapping-quality metrics of zero (e.g., MAPQ0), thereby causing the split-read alignment system 106 to filter out the candidate alignments of the mismatched transcriptomic read fragments 1118a. Because the split-read alignment system 106 filters out the mismatched transcriptomic read fragments 1118a aligned with a genomic region to one side of the breakpoint 1104a, the split-read alignment system 106 avoids determining a false-positive variant call of a gene-fusion event (as the existing sequencing system does) for the same genomic sample. By generating improved split group scores for candidate split alignments, the split-read alignment system 106 avoids the “noisy” split reads exhibited by the existing sequencing system's candidate alignments in the previous alignment window 1108a. Because it avoids such noisy split read alignments, the split-read alignment system 106 also avoids calling an incorrect gene-fusion variant and correctly identifies a true-negative variant for gene fusion.

FIGS. 11C and 11D complement one another by depicting a breakpoint along a chromosome (FIG. 11C) and different read fragment alignments and mappings determined by the split-read alignment system 106 and an existing sequencing system (FIG. 11D) with respect to the same breakpoint. As shown in FIG. 11C, for example, a chromosome segment 1102b for chromosome 4 comprises a breakpoint 1104b. In particular, the breakpoint 1104b shown in FIG. 11C identifies one or more genomic coordinates at which transcriptomic reads have been aligned by an existing sequencing system with a break between read fragments subsequently depicted in FIG. 11D. As further illustrated below, a split-read alignment of transcriptomic reads with respect to the breakpoint 1104b can indicate a gene-fusion event for the DCTD gene with another gene.

As shown in FIG. 11D, the user client device 108 presents a graphical user interface 1100b comprising different read fragment alignments and mappings determined by the split-read alignment system 106 and by an existing sequencing system with respect to the breakpoint 1104b. As above, for instance, the graphical user interface 1100b represents a graphical user interface from IGV comprising transcriptomic read alignments with respect to a reference genome. For purposes of comparison, the graphical user interface 1100b comprises an updated alignment window 1106b depicting transcriptomic read alignments of the split-read alignment system 106, a previous alignment window 1108b depicting transcriptomic read alignments of an existing sequencing system, and a reference-genome window 1110b depicting reference nucleotide bases of a reference genome. In FIG. 11D, the updated alignment window 1106b further comprises read coverage markers 1120b that indicate read coverage (e.g., read depth) at genomic coordinates overlapping with the breakpoint 1104b.

As shown in the previous alignment window 1108b, the existing sequencing system maps and aligns transcriptomic read fragments 1114b with the reference genome at genomic coordinates corresponding (or relatively closer) to the breakpoint 1104b. Similar to the graphical user interface 1100a in FIG. 11B, the graphical user interface 1100b in FIG. 11D includes light grey shading indicating that called nucleotide bases of transcriptomic read fragments (e.g., transcriptomic read fragments 1114b) match the reference nucleotide bases of the reference genome and different grey shading or colors indicating that called nucleotide bases of mismatched transcriptomic read fragments (e.g., mismatched transcriptomic read fragments 1112c, 1112d, and 1118b) do not much the reference nucleotide bases. In contrast to the transcriptomic read fragments 1114b, the existing sequencing system maps and aligns (i) mismatched transcriptomic read fragments 1112c with a genomic region corresponding to a contiguous sequence located upstream from the breakpoint 1104b and (ii) mismatched transcriptomic read fragments 1112d with a genomic region corresponding to a contiguous sequence located downstream from the breakpoint 1104b.

Because a threshold number of called nucleotide bases do not match the reference nucleotide bases, the existing sequencing system clips the nucleotide bases within the mismatched transcriptomic read fragments 1112c and 1112d, thereby ignoring the nucleotide bases of the mismatched transcriptomic read fragments 1112a and 1112b for purposes of alignment. As depicted in FIG. 11D, the mismatched transcriptomic read fragments 1112c and 1112d exhibit split alignments of corresponding transcriptomic reads within respect to the reference genome. Both the candidate alignments of the mismatched transcriptomic read fragments 1112c and 1112d by the existing sequencing system represent supplemental alignments with positive mapping-quality metrics (e.g., positive MAPQ) and correspond to primary alignments with another gene (not shown). Based on scoring of the primary and supplemental alignments of such corresponding transcriptomic reads depicted in the previous alignment window 1108b, the existing sequencing system determines a false-positive variant call of a gene-fusion event for a genomic sample. For instance, in some cases, the existing sequencing system re-aligns the mismatched transcriptomic read fragments 1112c and 1112d with genomic regions of another gene on a same chromosome (e.g., chromosome 4) or another gene on a different chromosome, thereby indicating a gene-fusion event.

As shown in the updated alignment window 1106b, by contrast, the split-read alignment system 106 maps and aligns mismatched transcriptomic read fragment 1118a with a genomic region corresponding to a contiguous sequence located upstream from the breakpoint 1104b, but does not map or align any mismatched transcriptomic read fragments downstream from the breakpoint 1104b. As further indicated by FIG. 11D, the candidate alignment of the mismatched transcriptomic read fragment 1118a by the split-read alignment system 106 exhibits a relatively low mapping-quality metric (e.g., MAPQ0), thereby causing the split-read alignment system 106 to filter out the candidate alignment of the mismatched transcriptomic read fragment 1118a. Because the split-read alignment system 106 filters out the mismatched transcriptomic read fragment 1118a aligned with a genomic region to one side of the breakpoint 1104b, the split-read alignment system 106 does not determine a false-positive variant call of a gene-fusion event for the same genomic sample. By generating improved split group scores for candidate split alignments, the split-read alignment system 106 avoids the “noisy” split reads exhibited by the existing sequencing system's candidate alignments in the previous alignment window 1108b. As above, because it avoids such noisy split read alignments, the split-read alignment system 106 also avoids calling an incorrect gene-fusion variant and correctly identifies a true-negative variant with respect to gene fusion.

In addition to improving the accuracy of mapping-and-alignment and variant calling for gene-fusion events, in some embodiments, the split-read alignment system 106 also improves nucleotide-read coverage and variant-calling accuracy for chromosome M for human mitochondrial DNA by selecting more accurate mapping and alignment based on improved split group scores. In accordance with one or more embodiments, FIGS. 12A-12D illustrate coverage graphs 1200a-1200d exhibiting higher coverage by nucleotide reads mapped and aligned to genomic regions of chromosome M using the split-read alignment system 106 relative to such coverage from nucleotide reads mapped and aligned using an existing sequencing system. As shown in FIGS. 12A-12D, the improved nucleotide-read coverage extends from the beginning of chromosome M to the more difficult-to-cover and difficult-to-call genomic regions at the end of chromosome M. In accordance with one or more embodiments, FIG. 13 illustrates a variant-call table 1300 exhibiting better accuracy for SNP calls and indel calls by the split-read alignment system 106 at genomic regions of chromosome M relative to such SNP calls and indel calls by an existing sequencing system.

The ending genomic regions of chromosome M are notoriously difficult to call and cover for existing sequencing systems in part due to the circular nature of mitochondrial DNA. Because existing models for mapping and aligning represent chromosome M's circular DNA in linear fashion, existing sequencing systems often chop up and incorrectly soft clip nucleotide reads that align with chromosome M's ending genomic regions and, therefore, sometimes incorrectly ignore valuable nucleotide-read data relevant to chromosome M's ending genomic regions. In contrast to existing sequencing systems and as exhibited by FIGS. 12A-12D, the split-read alignment system 106 generates improved split group scores that penalize split alignments across different chromosomes and thereby improves selection of split groups and mapping and alignment for chromosome M.

To test the nucleotide-read coverage for fragment alignments from the split-read alignment system 106, researchers executed the split-read alignment system 106 and an existing sequencing system on mitochondrial DNA samples from the Fazzini dataset, as described by Federica Fazzini et al., “Analyzing Low-Level mtDNA Heteroplasmy-Pitfalls and Challenges from Bench to Benchmarking,” Int'l J. Mol. Sci. 2021 Jan. 19; 22(2): 935, which is hereby incorporated by reference in its entirety. For instance, researchers sequenced and aligned nucleotide reads from two-person mtDNA mixtures with different target allele frequencies, where sample mixture M1 includes a 1:2 mixture and a target allele frequency of 50%, sample mixture M2 includes a 1:10 mixture and a target allele frequency of 10%, and sample mixture M3 includes a 1:50 mixture and a target allele frequency of 2%. In some cases, the researchers used different versions of Taq polymerase for a polymer chain reaction (PCR), including LA Advantage (by Clontech Laboratories), Herculase II Fusion (HERK), and LongAmp Taq Polymerase (NEB). The researchers also sequenced nucleotide reads from sample mixtures M1, M2, and M3 using two different protocols: PCR amplification before mixture and PCR after mixture. The researchers further plotted the nucleotide-read coverage at genomic coordinates at the beginning and ending of chromosome M in FIGS. 12A-12D. Additionally, the researchers determined false-positive and false-negative variant calls for SNPs and indels in sample mixtures M1, M2, and M3 using different versions of PCR Taq polymerase and protocols as depicted in FIG. 13.

As shown in FIGS. 12A and 12B, the coverage graphs 1200a and 1200b show coverage of nucleotide reads—sequenced from sample mixture M1 using HERK—that have been mapped and aligned by the split-read alignment system 106 and by an existing sequencing system. In FIGS. 12A and 12B, graph keys 1202a and 1202b display coverage plot lines for the split-read alignment system 106 designated as MapperV2 (i.e., MapperV2_All, MapperV2_60, MapperV2_20, and MapperV2_gvcf) and coverage plot lines for the existing sequencing system designated as curMapper (i.e., curMapper_All, curMapper_60, curMapper_20, and curMapper_gvcf). As indicated by the coverage graph 1200a and the graph key 1202a in FIG. 12A, the split-read alignment system 106 maps and aligns nucleotide reads with consistently higher coverage across the beginning genomic regions of chromosome M (chrM: 0-100) relative to the existing sequencing system, including all mapped nucleotide reads as shown by the comparison of plot lines for MapperV2_All and curMapper_All. Even more than the beginning genomic regions of chromosome M, as indicated by the coverage graph 1200b and the graph key 1202b in FIG. 12B, the split-read alignment system 106 maps and aligns nucleotide reads with consistently higher coverage across the ending genomic regions of chromosome M (chrM: 16469-16569) relative to the existing sequencing system, again including all mapped nucleotide reads as shown by the comparison of plot lines for MapperV2_All and curMapper_All.

As shown in FIGS. 12C and 12D, the coverage graphs 1200c and 1200d similarly show coverage of nucleotide reads—sequenced from sample mixture M1 using a Clontech Taq polymerase—that have been mapped and aligned by the split-read alignment system 106 and by an existing sequencing system. In FIGS. 12C and 12D, graph keys 1202c and 1202d display coverage plot lines for the split-read alignment system 106 designated as MapperV2 (i.e., MapperV2_All, MapperV2_60, MapperV2_20, and MapperV2_gvcf) and coverage plot lines for the existing sequencing system designated as curMapper (i.e., curMapper_All, curMapper_60, curMapper_20, and curMapper_gvcf). As indicated by the coverage graph 1200c and the graph key 1202c in FIG. 12C, the split-read alignment system 106 maps and aligns nucleotide reads with consistently higher coverage across the beginning genomic regions of chromosome M (chrM: 0-100) relative to the existing sequencing system, including all mapped nucleotide reads as shown by the comparison of plot lines for MapperV2_All and curMapper_All. Even more than the beginning genomic regions of chromosome M, as indicated by the coverage graph 1200d and the graph key 1202d in FIG. 12D, the split-read alignment system 106 again maps and aligns nucleotide reads with consistently higher coverage across the ending genomic regions of chromosome M (chrM: 16469-16569) relative to the existing sequencing system, again including all mapped nucleotide reads as shown by the comparison of plot lines for MapperV2_All and curMapper_All.

As indicated above, FIG. 13 depicts the variant-call table 1300 showing false-positive and false-negative variant calls for SNPs and indels by the split-read alignment system 106 and an existing sequencing system at genomic regions of chromosome M for sample mixtures M1, M2, and M3 using different versions of PCR Taq polymerase and different PCR protocols. On the left side, the variant-call table 1300 shows false-positive and false-negative variant calls for SNPs and indels by the existing sequencing system, as indicated by the column for “Datasetjama_REV7169.” On the right side, the variant-call table 1300 shows false-positive and false-negative variant calls for SNPs and indels by the split-read alignment system 106, as indicated by the column for “CGM_mapperV2.” As indicated by the “Total” and “Diff” columns of the variant-call table 1300, the split-read alignment system 106 consistently determines fewer total false-positive and false-negative SNP and indel calls than the existing sequencing system. While the “Diff” column of the variant-call table 1300 shows the split-read alignment system 106 exhibits between one and eight fewer false-positive and false-negative SNP and indel calls, such a reduction in false-positive and false-negative SNP and indel calls is significant for such a short chromosome—that is, chromosome M of only 16,569 base-pairs long.

Beyond improved nucleotide-read coverage and improved variant calling for chromosome M, in some embodiments, the split-read alignment system 106 also improves the accuracy of structural variant calls. In accordance with one or more embodiments, FIG. 14A depicts a table 1400a showing the split-read alignment system 106 recovering insertion calls that an existing sequencing system missed for a gene that affects acute myeloid leukemia (AML). In accordance with one or more embodiments, FIG. 14B depicts a table 1400b showing the split-read alignment system 106 determine more accurate duplication and translocation calls relative to the existing sequencing system.

As shown in FIG. 14A, for example, the table 1400a compares insertion calls by the split-read alignment system 106 and an existing sequencing system within the fms-like tyrosine kinase 3 (FLT3) gene for known genomic samples from both normal and tumor tissues. Mutations of the FLT3 gene are responsible for a significant percentage of AML cases, with the internal tandem duplication (ITD) representing the most common type of FLT3 mutation. As indicated by the table 1400a, based on improved split group scores and selecting better improved split groups for variant calling, the split-read alignment system 106 (shown in the column for “new M/A+ Call Generation Model”) correctly determines insertion calls over at least 50 base-pairs long at genomic coordinates chr13:28034103 and chr13:28034120 for a couple of known genomic samples with FLT3-ITD mutations, but the existing sequencing system (shown in the column for “Call Generation Model” only) miscalls such insertions. As further indicated by the table 1400a, the split-read alignment system 106 (shown in the column for “new M/A+ Call Generation Model”) also correctly determines the presence or absence of such insertions at other genomic coordinates that the existing sequencing system (shown in the column for “Call Generation Model” only) also correctly determined. Such recovered insertion calls (and retention of previously accurate insertion calls) by the split-read alignment system 106 demonstrates critical accuracy improvements and accuracy retention for structural variant calls within a gene important to cancer diagnoses.

As shown in FIG. 14B, the table 1400b compares the accuracy of somatic structural variant calls by the split-read alignment system 106 and by an existing sequencing system from sequencing data HCC1954. HCC1954 is a cell line exhibiting epithelial breast cancer. As indicated by the table 1400b, based on improved split group scores and selecting better improved split groups for variant calling, the split-read alignment system 106 (shown in the rows for “new M/A+ Call Generation Model”) exhibits better recall, precision, and F-score for duplication calls in HCC1954 than the existing sequencing system (shown in the rows for “Call Generation Model” only). As also indicated by the table 1400b, the split-read alignment system 106 (shown in the rows for “new M/A+ Call Generation Model”) exhibits better precision and F-score for translocation calls in HCC1954 than the existing sequencing system (shown in the rows for “Call Generation Model”). As the recall, precision, and F-scores reported in the table 1400b were determined without ground-truth calls, the recall, precision, and F-scores for the split-read alignment system 106 when determined with ground-truth calls.

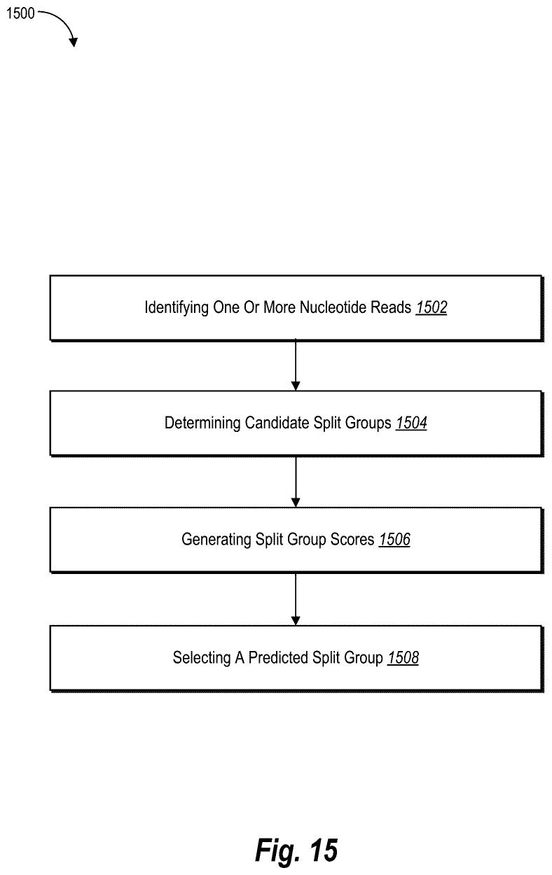

FIGS. 1-14B the corresponding text, and the examples provide a number of different methods, systems, devices, and non-transitory computer-readable media of the split-read alignment system 106. In addition to the foregoing, one or more implementations can also be described in terms of flowcharts comprising acts for accomplishing a particular result, as shown in FIG. 15. FIG. 15 may be performed with more or fewer acts. Further, the acts may be performed in differing orders. Additionally, the acts described herein may be repeated or performed in parallel with one another or parallel with different instances of the same or similar acts.

As mentioned, FIG. 15 illustrates a flowchart of a series of acts 1500 for selecting a predicted split group from candidate split groups. While FIG. 15 illustrates acts according to one implementation, alternative implementations may omit, add to, reorder, and/or modify any of the acts shown in FIG. 15. The acts of FIG. 15 can be performed as part of a method. Alternatively, a non-transitory computer-readable medium can comprise instructions that, when executed by at least one processor, cause a computing device to perform the acts of FIG. 15. In still further embodiments, a system comprising at least one processor and a non-transitory computer readable medium comprising instructions that, when executed by one or more processors, cause the system to perform the acts of FIG. 15. In some cases, the at least one processor comprises a configurable processor and executing the at least one processor comprises configuring the configurable processor.

As shown in FIG. 15, the series of acts 1500 includes an act 1502 of identifying one or more nucleotide reads. In particular, the act 1502 comprises identifying one or more nucleotide reads corresponding to a genomic region of a genomic sample.

The series of acts 1500 illustrated in FIG. 15 further includes an act 1504 of determining candidate split groups. In particular, the act 1504 comprises determining candidate split groups comprising fragment alignments corresponding to the one or more nucleotide reads. In some implementations, determining a candidate split group of the candidate split groups further comprises grouping, into the candidate split group, one or more fragment alignments of a single-end nucleotide read; or grouping, into the candidate split group, one or more fragment alignments of a paired-end nucleotide read from a pair of paired-end nucleotide reads.

As further illustrated in FIG. 15, the series of acts 1500 includes an act 1506 of generating split group scores. In particular, the act 1506 comprises generating split group scores for split alignments of the candidate split groups with a reference genome. In some implementations, the act 1506 further comprises additional acts of generating fragment alignment scores for individual fragment alignments of a candidate split group with the reference genome; and generating a split group score for the candidate split group based on the fragment alignment scores. Additionally, in some implementations, the act 1506 further comprises generating, for a candidate split group of the candidate split groups, a break penalty for relative geometries of a first fragment alignment and a second fragment alignment with respect to the reference genome; and generating a split group score for the candidate split group based on the break penalty. Furthermore, in some implementations, the series of acts 1506 comprises generating, for a candidate split group of the candidate split groups, an overlap penalty for an overlap within a nucleotide read between a first fragment alignment and a second fragment alignment; and generating a split group score for the candidate split group based on the overlap penalty.

In some embodiments, the act 1506 further comprises generating a split group score for a candidate split group of the candidate split groups by: generating fragment alignment scores, a break penalty, and an overlap penalty for fragment alignments of the candidate split group; and combining the fragment alignment scores and subtracting the break penalty and the overlap penalty from the combined fragment alignment scores. In some implementations, the act 1006 further comprises determining the candidate split groups by iteratively grouping individual fragment alignments following an order of outermost fragment alignments to innermost fragment alignments of a nucleotide read; and generating the split group scores by iteratively scoring groupings of individual fragment alignments following the order in which the individual fragment alignments were grouped.

The series of acts 1500 illustrated in FIG. 15 further includes an act 1508 of selecting a predicted split group. In particular, the act 1508 comprises selecting, for nucleobase calling of the genomic region, a predicted split group from the candidate split groups based on the split group scores. In some embodiments, the act 1508 comprises identifying, from the candidate split groups, candidate pairs of split groups comprising different fragment alignments for mates of a paired-end nucleotide read; generating, for the candidate pairs of split groups, pair scores evaluating pair alignments of the candidate pairs of split groups with the reference genome; and selecting, for each mate of the paired-end nucleotide read, the predicted split group based further on the pair scores. Furthermore, in some embodiments, the act 1508 further comprises determining sums of split group scores for respective candidate pairs of split groups; generating pairing penalties based on an estimated insert size between innermost fragment alignments of the candidate pairs of split groups; and generating the pair scores for the candidate pairs of split groups based on the sums of split group scores and the pairing penalties.

In some embodiments, the series of acts 1500 includes additional acts of determining an alt-contig fragment alignment score for an inner fragment alignment and an outer fragment alignment corresponding to a nucleotide read with an alternate contiguous sequence within the reference genome; determining a split group score for the inner fragment alignment and the outer fragment alignment with a primary-assembly region of the reference genome; and selecting the alt-contig fragment alignment score as a replacement split group score based on determining that the alt-contig fragment alignment score exceeds the split group score.

Additionally, in one or more implementations, the series of acts 1500 includes an additional act of determining nucleobase calls for the genomic region based on an alignment of the predicted split group with the reference genome.

The series of acts 1500 may also include additional acts of determining that a fragment alignment score of a fragment alignment fails to satisfy a threshold fragment alignment score; and removing the fragment alignment from consideration in forming the candidate split group.

The series of acts 1500 may include additional acts of determining that an alignment score for a candidate split group fails to satisfy a minimum alignment score; and refraining from reporting a split alignment of the candidate split group in an alignment file or a variant call file based on the alignment score failing to satisfy the minimum alignment score.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Implementations in which images are obtained in different color channels, for example, coinciding with different labels used to distinguish one nucleobase type from another are particularly applicable. In some implementations, the process to determine the nucleotide sequence of a target nucleic acid (i.e., a nucleic-acid polymer) can be an automated process. Preferred implementations include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as the release of pyrophosphate; or the like. In implementations, where two or more different nucleotides are present in a sequencing reagent, the different nucleotides can be distinguishable from each other, or alternatively, the two or more different labels can be indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred implementations include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to the incorporation of nucleotides at the features of the array. An image can be obtained after the array is treated with a particular nucleotide type (e.g., A, T, C, or G). Images obtained after the addition of each nucleotide type will differ with regard to which features in the array are detected. These differences in the image reflect the different sequence content of the features on the array. However, the relative locations of each feature will remain unchanged in the images. The images can be stored, processed, and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed, and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing implementations, the labels do not substantially inhibit extension under SBS reaction conditions. However, the detection labels can be removable, for example, by cleavage or degradation. Images can be captured following the incorporation of labels into arrayed nucleic acid features. In particular implementations, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such implementations, each image will show nucleic acid features that have incorporated nucleotides of a particular type. Different features are present or absent in the different images due to the different sequence content of each feature. However, the relative position of the features will remain unchanged in the images. Images obtained from such reversible terminator-SBS methods can be stored, processed, and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular implementations, some or all of the nucleotide monomers can include reversible terminators. In such implementations, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30-second exposure to long-wavelength UV light. Thus, either disulfide reduction or photocleavage can be used as a cleavable linker. Another approach to reversible termination is the use of natural termination that ensues after the placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some implementations can utilize the detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes an apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on the presence of their respective signals and incorporation of the fourth nucleotide type into the nucleic acid can be determined based on the absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary implementation that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some implementations can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due to the different sequence content of each feature, but the relative position of the features will remain unchanged in the images. Images obtained from ligation-based sequencing methods can be stored, processed, and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some implementations can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such implementations, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed, and analyzed as set forth herein. In particular, the data can be treated as an image in accordance with the exemplary treatment of optical images and other images that is set forth herein.

Some implementations can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed, and analyzed as set forth herein.

Some SBS implementations include the detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular implementations, different target nucleic acids can be treated in a common reaction vessel or on a surface of a particular substrate. This allows convenient delivery of sequencing reagents, removal of unreacted reagents, and detection of incorporation events in a multiplex manner. In implementations using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle, or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines, and the like. A flow cell can be configured and/or used in an integrated system for the detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing implementation as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic-acid polymers present in samples received by a sequencing device. As defined herein, a “sample” (and its derivatives) is used in its broadest sense and includes any specimen, culture, and the like that is suspected of including a target. In some implementations, the sample comprises DNA, RNA, PNA, LNA, chimeric or hybrid forms of nucleic acids. The sample can include any biological, clinical, surgical, agricultural, atmospheric, or aquatic-based specimen containing one or more nucleic acids. The term also includes any isolated nucleic acid sample such a genomic DNA, fresh-frozen, or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample, and normal tissue sample, or sample from a single source that contains two distinct forms of genetic material such as maternal and fetal DNA obtained from a maternal subject, or the presence of contaminating bacterial DNA in a sample that contains plant or animal DNA. In some implementations, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another implementation, low molecular weight material includes enzymatically or mechanically fragmented DNA. The sample can include cell-free circulating DNA. In some implementations, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some implementations, the sample can be an epidemiological, agricultural, forensic, or pathogenic sample. In some implementations, the sample can include nucleic acid molecules obtained from an animal such as a human or mammalian source. In another implementation, the sample can include nucleic acid molecules obtained from a non-mammalian source such as a plant, bacteria, virus, or fungus. In some implementations, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one implementation, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example, derived from a buccal swab, paper, fabric, or other substrates that may be impregnated with saliva, blood, or other bodily fluids. As such, in some implementations, the nucleic acid sample may comprise low amounts of, or fragmented portions of DNA, such as genomic DNA. In some implementations, target sequences can be present in one or more bodily fluids including but not limited to, blood, sputum, plasma, semen, urine, and serum. In some implementations, target sequences can be obtained from hair, skin, tissue samples, autopsy or remains of a victim. In some implementations, nucleic acids including one or more target sequences can be obtained from a deceased animal or human. In some implementations, target sequences can include nucleic acids obtained from non-human DNA such a microbial, plant, or entomological DNA. In some implementations, target sequences or amplified target sequences are directed to purposes of human identification. In some implementations, the disclosure relates generally to methods for identifying characteristics of a forensic sample. In some implementations, the disclosure relates generally to human identification methods using one or more target-specific primers disclosed herein or one or more target-specific primers designed using the primer design criteria outlined herein. In one implementation, a forensic or human identification sample containing at least one target sequence can be amplified using any one or more of the target-specific primers disclosed herein or using the primer criteria outlined herein.

The components of the split-read alignment system 106 can include software, hardware, or both. For example, the components of the split-read alignment system 106 can include one or more instructions stored on a computer-readable storage medium and executable by processors of one or more computing devices (e.g., the user client device 108). When executed by the one or more processors, the computer-executable instructions of the split-read alignment system 106 can cause the computing devices to perform the bubble detection methods described herein. Alternatively, the components of the split-read alignment system 106 can comprise hardware, such as special-purpose processing devices to perform a certain function or group of functions. Additionally, or in the alternative, the components of the split-read alignment system 106 can include a combination of computer-executable instructions and hardware.

Furthermore, the components of the split-read alignment system 106 performing the functions described herein with respect to the split-read alignment system 106 may, for example, be implemented as part of a stand-alone application, as a module of an application, as a plug-in for applications, as a library function or functions that may be called by other applications, and/or as a cloud-computing model. Thus, components of the split-read alignment system 106 may be implemented as part of a stand-alone application on a personal computing device or a mobile device. Additionally, or alternatively, the components of the split-read alignment system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Implementations of the present disclosure may comprise or utilize a special purpose or general-purpose computer including computer hardware, such as, for example, one or more processors and system memory, as discussed in greater detail below. Implementations within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general-purpose or special-purpose computer system. Computer-readable media that store computer-executable instructions are non-transitory computer-readable storage media (devices). Computer-readable media that carry computer-executable instructions are transmission media. Thus, by way of example, and not limitation, implementations of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid-state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general-purpose or special-purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links that can be used to carry desired program code means in the form of computer-executable instructions or data structures and which can be accessed by a general-purpose or special-purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some implementations, computer-executable instructions are executed on a general-purpose computer to turn the general-purpose computer into a special-purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Implementations of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 16 illustrates a block diagram of a computing device 1600 that may be configured to perform one or more of the processes described above. One will appreciate that one or more computing devices such as the computing device 1600 may implement the split-read alignment system 106 and the sequencing system 104. As shown by FIG. 16, the computing device 1600 can comprise a processor 1602, a memory 1604, a storage device 1606, an I/O interface 1608, and a communication interface 1610, which may be communicatively coupled by way of a communication infrastructure 1612. In certain implementations, the computing device 1600 can include fewer or more components than those shown in FIG. 16. The following paragraphs describe components of the computing device 1600 shown in FIG. 16 in additional detail.

In one or more implementations, the processor 1602 includes hardware for executing instructions, such as those making up a computer program. As an example, and not by way of limitation, to execute instructions for dynamically modifying workflows, the processor 1602 may retrieve (or fetch) the instructions from an internal register, an internal cache, the memory 1604, or the storage device 1606 and decode and execute them. The memory 1604 may be a volatile or non-volatile memory used for storing data, metadata, and programs for execution by the processor(s). The storage device 1606 includes storage, such as a hard disk, flash disk drive, or another digital storage device, for storing data or instructions for performing the methods described herein.

The I/O interface 1608 allows a user to provide input to, receive output from, and otherwise transfer data to and receive data from computing device 1600. The I/O interface 1608 may include a mouse, a keypad or a keyboard, a touch screen, a camera, an optical scanner, network interface, modem, other known I/O devices, or a combination of such I/O interfaces. The I/O interface 1608 may include one or more devices for presenting output to a user, including, but not limited to, a graphics engine, a display (e.g., a display screen), one or more output drivers (e.g., display drivers), one or more audio speakers, and one or more audio drivers. In certain implementations, the I/O interface 1608 is configured to provide graphical data to a display for presentation to a user. The graphical data may be representative of one or more graphical user interfaces and/or any other graphical content as may serve a particular implementation.

The communication interface 1610 can include hardware, software, or both. In any event, the communication interface 1610 can provide one or more interfaces for communication (such as, for example, packet-based communication) between the computing device 1600 and one or more other computing devices or networks. As an example, and not by way of limitation, the communication interface 1610 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1610 may facilitate communications with various types of wired or wireless networks. The communication interface 1610 may also facilitate communications using various communication protocols. The communication infrastructure 1612 may also include hardware, software, or both that couples components of the computing device 1600 to each other. For example, the communication interface 1610 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary implementations thereof. Various implementations and aspects of the present disclosure(s) are described with reference to details discussed herein, and the accompanying drawings illustrate the various implementations. The description above and drawings are illustrative of the disclosure and are not to be construed as limiting the disclosure. Numerous specific details are described to provide a thorough understanding of various implementations of the present disclosure.

The present disclosure may be embodied in other specific forms without departing from its spirit or essential characteristics. The described implementations are to be considered in all respects only as illustrative and not restrictive. For example, the methods described herein may be performed with fewer or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

What is claimed is:

1. A computer-implemented method comprising:
identifying one or more nucleotide reads corresponding to a genomic region of a genomic sample;
determining candidate split groups comprising fragment alignments corresponding to the one or more nucleotide reads;
generating split group scores for split alignments of the candidate split groups with a reference genome; and
selecting, for nucleobase calling of the genomic region, a predicted split group from the candidate split groups based on the split group scores.

2. The computer-implemented method of claim 1, further comprising determining a candidate split group of the candidate split groups by:
grouping, into the candidate split group, one or more fragment alignments of a single-end nucleotide read; or
grouping, into the candidate split group, one or more fragment alignments of a paired-end nucleotide read from a pair of paired-end nucleotide reads.

3. The computer-implemented method of claim 1, further comprising:
generating fragment alignment scores for individual fragment alignments of a candidate split group with the reference genome; and
generating a split group score for the candidate split group based on the fragment alignment scores.

4. The computer-implemented method of claim 1, further comprising:
generating, for a candidate split group of the candidate split groups, a break penalty for relative geometries of a first fragment alignment and a second fragment alignment with respect to the reference genome; and
generating a split group score for the candidate split group based on the break penalty.

5. The computer-implemented method of claim 1, further comprising:
generate, for a candidate split group of the candidate split groups, an overlap penalty for an overlap within a nucleotide read between a first fragment alignment and a second fragment alignment; and
generate a split group score for the candidate split group based on the overlap penalty.

6. The computer-implemented method of claim 1, further comprising generating a split group score for a candidate split group of the candidate split groups by:
generating fragment alignment scores, a break penalty, and an overlap penalty for fragment alignments of the candidate split group; and
combining the fragment alignment scores and subtracting the break penalty and the overlap penalty from the combined fragment alignment scores.

7. The computer-implemented method of claim 1, further comprising:
determining the candidate split groups by iteratively grouping individual fragment alignments following an order of outermost fragment alignments to innermost fragment alignments of a nucleotide read; and
generating the split group scores by iteratively scoring groupings of individual fragment alignments following the order in which the individual fragment alignments were grouped.

8. The computer-implemented method of claim 1, further comprising:
identifying, from the candidate split groups, candidate pairs of split groups comprising different fragment alignments for mates of a paired-end nucleotide read;
generating, for the candidate pairs of split groups, pair scores evaluating pair alignments of the candidate pairs of split groups with the reference genome; and
selecting, for each mate of the paired-end nucleotide read, the predicted split group based further on the pair scores.

9. The computer-implemented method of claim 8, further comprising:
determining sums of split group scores for respective candidate pairs of split groups;
generating pairing penalties based on an estimated insert size between innermost fragment alignments of the candidate pairs of split groups; and
generating the pair scores for the candidate pairs of split groups based on the sums of split group scores and the pairing penalties.

10. The computer-implemented method of claim 8, further comprising:
determining an alt-contig fragment alignment score for an inner fragment alignment and an outer fragment alignment corresponding to a nucleotide read with an alternate contiguous sequence within the reference genome;
determining a split group score for the inner fragment alignment and the outer fragment alignment with a primary-assembly region of the reference genome; and
selecting the alt-contig fragment alignment score as a replacement split group score based on determining that the alt-contig fragment alignment score exceeds the split group score.

11. A system comprising:
at least one processor; and
a non-transitory computer-readable medium comprising instructions that, when executed by the at least one processor, cause the system to:
identify one or more nucleotide reads corresponding to a genomic region of a genomic sample;
determine candidate split groups comprising fragment alignments corresponding to the one or more nucleotide reads;
generate split group scores for split alignments of the candidate split groups with a reference genome; and
select, for nucleobase calling of the genomic region, a predicted split group from the candidate split groups based on the split group scores.

12. The system of claim 11, further comprising instructions that, when executed by the at least one processor, cause the system to determine nucleobase calls for the genomic region based on an alignment of the predicted split group with the reference genome.

13. The system of claim 11, further comprising instructions that, when executed by the at least one processor, cause the system to:
determine that a fragment alignment score of a fragment alignment fails to satisfy a threshold fragment alignment score; and
remove the fragment alignment from consideration in forming the candidate split groups.

14. The system of claim 11, further comprising instructions that, when executed by the at least one processor, cause the system to:
determine that an alignment score for a candidate split group fails to satisfy a minimum alignment score; and
refrain from reporting a split alignment of the candidate split group in an alignment file or a variant call file based on the alignment score failing to satisfy the minimum alignment score.

15. The system of claim 11, further comprising instructions that, when executed by the at least one processor, cause the system to generate a split group score for a candidate split group of the candidate split groups by:
generating fragment alignment scores, a break penalty, and an overlap penalty for fragment alignments of the candidate split group; and
combining the fragment alignment scores and subtracting the break penalty and the overlap penalty from the combined fragment alignment scores.

16. The system of claim 11, further comprising instructions that, when executed by the at least one processor, cause the system to:
determine the candidate split groups by iteratively grouping individual fragment alignments following an order of outermost fragment alignments to innermost fragment alignments of a nucleotide read; and
generate the split group scores by iteratively scoring groupings of individual fragment alignments following the order in which the individual fragment alignments were grouped.

17. A non-transitory computer-readable medium comprising instructions that, when executed by at least one processor, cause a computing device to:
identify one or more nucleotide reads corresponding to a genomic region of a genomic sample;
determine candidate split groups comprising fragment alignments corresponding to the one or more nucleotide reads;
generate split group scores for split alignments of the candidate split groups with a reference genome; and
select, for nucleobase calling of the genomic region, a predicted split group from the candidate split groups based on the split group scores.

18. The non-transitory computer-readable medium of claim 17, further comprising instructions that, when executed by the at least one processor, cause the computing device to determine a candidate split group of the candidate split groups by:
grouping, into the candidate split group, one or more fragment alignments of a single-end nucleotide read; or
grouping, into the candidate split group, one or more fragment alignments of a paired-end nucleotide read from a pair of paired-end nucleotide reads.

19. The non-transitory computer-readable medium of claim 17, further comprising instructions that, when executed by the at least one processor, cause the computing device to:
generate fragment alignment scores for individual fragment alignments of a candidate split group with the reference genome; and
generate a split group score for the candidate split group based on the fragment alignment scores.

20. The non-transitory computer-readable medium of claim 17, further comprising instructions that, when executed by the at least one processor, cause the computing device to:
generate, for a candidate split group of the candidate split groups, a break penalty for relative geometries of a first fragment alignment and a second fragment alignment with respect to the reference genome; and
generate a split group score for the candidate split group based on the break penalty.
